SEQUENCE LISTING (4 sequences)

SEQ ID NO: 1 - protein, 5 aa, Artificial Sequence
  Description of Artificial Sequence; note=synthetic construct
TFLLR

SEQ ID NO: 2 - DNA, 18 bp, Artificial Sequence
  Description of Artificial Sequence; note=synthetic construct
gaagggacttgctattgg

SEQ ID NO: 3 - DNA, 21 bp, Artificial Sequence
  Description of Artificial Sequence; note=synthetic construct
gctcttcagcaatatcacggg

SEQ ID NO: 4 - protein, 12 aa, Artificial Sequence
  Description of Artificial Sequence; note=synthetic construct
MEQKLISEEDLN

(19) United States
(12) Patent Application Publication
(10) Pub. No.: US 2004/0096443 A1
(43) Pub. Date: May 20, 2004

(54) Treatment of neurodegenerative diseases and conditions using par1 antagonists

(76) Inventors: Stephen Francis Traynelis, Decatur, GA (US); Candice Elaine Junge, Santa Monica, CA (US); Robert J McKeon, Atlanta, GA (US); Melissa Butler Gingrich, San Diego, CA (US)

Correspondence Address: NEEDLE & ROSENBERG, P.C., SUITE 1000, 999 PEACHTREE STREET, ATLANTA, GA, 30309-3915, US

(21) Appl. No.: 10/471,032

(22) PCT Filed: Mar. 8, 2002
(86) PCT No.: PCT/US02/07280

(51) Int. Cl. A61K 39/395

(52) U.S. Cl. 424/143.1

(57) ABSTRACT

The present invention features methods for treating and/or preventing neurodegenerative and/or neurotoxic diseases, conditions, and injuries, by inhibiting the activity of protease-activated receptor-1 (PAR1) on neurons and glial cells using PARI antagonists.

### CROSS REFERENCE TO RELATED APPLICATIONS

This application claims benefit of priority from U.S. Provisional Application Serial No. 60/274,189, filed Mar. 8, 2001, which application is hereby incorporated by reference in its entirety.

### STATEMENT OF FEDERALLY SPONSORED RESEARCH

This invention was made with government support under Grant Nos. NS33777 and NS39419 awarded by the National Institute of Neurological Disorders and Stroke, National Institutes of Health. The government has certain rights in the invention.

### FIELD OF THE INVENTION

This invention relates generally to inhibition of central nervous system damage by neurodegenerative and/or neurotoxic diseases, conditions, and injuries, by inhibiting the activity of protease-activated receptor 1 (PAR1), which is also known as the thrombin receptor.

### BACKGROUND OF THE INVENTION

The protease-activated receptor 1 (PAR1, also known as the thrombin receptor) is activated by thrombin, a serine protease that is generated by proteolysis of its precursor, prothrombin, at sites of vascular injury. Thrombin-mediated stimulation of platelets via PAR1 is an important step in clot formation and wound healing in blood vessels.

Thrombin activates PAR1 by binding to the PAR1 anion-binding exosite and proteolytically cleaving the extracellular N-terminal domain of PAR1 at Arg41; this cleavage step releases a small peptide and unmasks a new PAR1 N-terminus. The first few amino acids (SFLLRN) of the new PAR1 N-terminus act as a tethered ligand that binds to another part of the receptor to initiate signaling by an associated G-protein. PAR1 can also be activated, to a lesser degree, by plasmin and other serine proteases involved in blood clotting.

Although PAR1 is widely distributed in neurons and glia, thrombin and other serine proteases involved in the clotting cascade are normally excluded from brain tissue by the blood-brain barrier. However, head trauma, stroke, status epilepticus, and other neuropathological conditions can compromise the integrity of the blood-brain barrier, thereby allowing blood proteins to gain access to the intracellular spaces that surround neurons and glia. Breakdown of the blood-brain barrier due to neurodegenerative diseases, conditions, or injuries can lead to undesirable consequences, such as glial scarring, edema, seizure, and/or neuronal death. Therefore, there is a need in the art for therapies that can reduce the severity of these undesirable consequences of neurodegenerative and/or neurotoxic diseases, conditions, and injuries.

### SUMMARY OF THE INVENTION

A broad variety of neurodegenerative and/or neurotoxic diseases, conditions, and injuries can cause neuronal death and/or reactive gliosis/glial scar formation. The present invention is based on the discovery that reduction or inhibition of PAR1/thrombin receptor activity in neurons and glia decreases the level of neuronal death and/or gliotic scar formation in the central nervous system of individuals who have, or who are at risk for having, a neurodegenerative or neurotoxic disease, condition, or injury, relative to the level of neuronal death and/or gliotic scar formation that would have occurred had there been no inhibition of PAR1/thrombin receptor activity. Accordingly, the present invention is useful for treating any disease, condition, or injury that places a patient at risk for neuronal death and/or glial scar formation, e.g., by lessening the severity of the neurodegenerative/neurotoxic effects of the disease, condition, or injury and/or improving the likelihood for recovery.

In a first aspect, the invention features a method of treating a patient with an injury of the central nervous system caused by a trauma to the head or spinal cord, comprising administering to the patient a therapeutically effective amount of a PAR1/thrombin receptor antagonist, thereby treating the injury to the central nervous system caused by the trauma to the head or spinal cord.

For example, the injury can be, but is not limited to, a penetrating injury, a crush injury, a compression injury, a stretch injury, or a blunt injury.

In a second aspect, the invention features a method of treating a patient with a subarachnoid hemorrhage, comprising administering to the patient a therapeutically effective amount of a PAR1/thrombin receptor antagonist, thereby treating the subarachnoid hemorrhage.

In a third aspect, the invention features a method of inhibiting reactive gliosis and/or gliotic scar formation in a patient in need thereof, comprising: a) identifying the patient as being in need of administration of a therapeutically effective amount of a PAR1/thrombin receptor antagonist for the purpose of inhibiting gliosis and/or gliotic scar formation, and b) administering to the patient a therapeutically effective amount of the PAR1/thrombin receptor antagonist, thereby inhibiting the gliosis and/or gliotic scar formation.

In a fourth aspect, the invention features a method of reducing damage to the brain as a result of intracerebral bleeding, comprising: a) identifying a patient as being in need of administration of a therapeutically effective amount of a PAR1/thrombin receptor antagonist for the purpose of reducing damage to the brain as a result of intracerebral bleeding, and b) administering to the patient a therapeutically effective amount of the PAR1/thrombin receptor antagonist, thereby reducing damage to the brain as a result of intracerebral bleeding.

For example, the intracerebral bleeding can be, but is not limited to, the result of a brain aneurysm, ruptured arteriovenous malformation, or traumatic brain injury.

In a fifth aspect, the invention features a method of reducing brain damage or neuron death that results from a seizure, comprising administering, to a patient who is having a seizure, who has recently had a seizure, or who is at risk for having a seizure, a therapeutically effective amount of a PAR1/thrombin receptor antagonist, thereby reducing brain damage that results from a seizure.

For example, the seizure can be associated with chronic epilepsy, idiopathic epilepsy, post-traumatic epilepsy, or status epilepticus.

In a sixth aspect, the invention features a method of reducing neuron death that is caused by NMDA receptor overactivation in a patient, comprising: a) identifying a patient as being in need of administration of a therapeutically effective amount of a PAR1/thrombin receptor antagonist for the purpose of reducing neuron death that is caused by NMDA receptor overactivation, and b) administering to the patient a therapeutically effective amount of the PAR1/thrombin receptor antagonist, thereby reducing neuron death that is caused by NMDA receptor overactivation in the patient.

In a seventh aspect, the invention features a method of treating a neuropathological disease, condition, or injury, comprising: a) identifying a subject as being in need of treatment to reduce neuron death by inhibition of PAR1 receptor activity on neurons, or as being in need of treatment to reduce reactive gliosis and/or glial scar formation by inhibition of PAR1 receptor activity on glia, and b) administering to the patient a therapeutically effective amount of a PAR1/thrombin receptor antagonist, thereby treating the neurological disease, condition, or injury.

For example, the neuropathological disease, condition, or injury can be, but is not limited to, head trauma, spinal cord trauma, brain aneurysm, intracerebral bleeding, subarachnoid hemorrhage, seizure, idiopathic epilepsy, chronic epilepsy, post-traumatic epilepsy, status epilepticus, Alzheimer's disease, Parkinson's disease, lower spinal motor neuron disease, upper motor neuron disease, amyotrophic lateral sclerosis, multiple sclerosis, or demyelinating disease.

The neuropathological disease, condition, or injury can also be occlusive stroke, hemorrhagic stroke, thrombosis, stenosis, or transient ischemic attack.

In an eighth aspect, the invention features a method of treating a patient during or following global ischemia as a result of cardiac surgery or pulmonary surgery, comprising administering to the patient a therapeutically effective amount of a PAR1/thrombin receptor antagonist for the purpose of: a) reducing brain damage, gliosis, gliotic scar formation, and/or neuronal death resulting from global ischemia resulting from cardiac surgery or pulmonary surgery, and/or b) facilitating recovery from neurological deficit resulting from cardiac surgery or pulmonary surgery, thereby treating a patient following or during global ischemia as a result of cardiac surgery or pulmonary surgery.

In a ninth aspect, the invention features a method of treating a patient suffering from a hypoxic condition, comprising administering to the patient a therapeutically effective amount of a PAR1/thrombin receptor antagonist for the purpose of: a) reducing the associated brain damage, gliosis, gliotic scar formation, and/or neuronal death resulting from the hypoxic condition, and/or b) facilitating recovery from neurological deficit resulting from the hypoxic condition, thereby treating the patient suffering from a hypoxic condition.

For example, the hypoxic condition can be anemic hypoxia, hypoxic hypoxia, anoxic hypoxia, histopathic hypoxia, or hypoglycemic hypoxia.

In a tenth aspect, the invention features a method of treating a patient with hypoglycemia, comprising administering to the patient a therapeutically effective amount of a PAR1/thrombin receptor antagonist for the purpose of: a) reducing brain damage, gliosis, gliotic scar formation, and/or neuronal death resulting from hypoglycemia, and/or b) facilitating recovery from neurological deficit resulting from hypoglycemia, thereby treating the patient with hypoglycemia.

In an eleventh aspect, the invention features a method of treating a patient with a bacterial, viral, or fungal infection of the central nervous system, comprising administering to the patient a therapeutically effective amount of a PAR1/thrombin receptor antagonist as an adjunct treatment for the purpose of: a) reducing brain damage, gliosis, gliotic scar formation, and/or neuronal death resulting from the infection, and/or b) facilitating recovery from neurological deficit resulting from the infection, thereby treating the patient with a bacterial, viral, or fungal infection of the central nervous system.

In a twelfth aspect, the invention features a method of treating a patient with a prion infection, comprising administering to the patient a therapeutically effective amount of a PAR1/thrombin receptor antagonist as an adjunct treatment for the purpose of: a) reducing brain damage, gliosis, gliotic scar formation, and/or neuronal death resulting from the prion infection, and/or b) facilitating recovery from neurological deficit resulting from the prion infection, thereby treating the patient with a prion infection.

For example, the patient can be suffering from Creutzfeldt-Jakob disease or other diseases associated with prion infections.

In a thirteenth aspect, the invention features a method of treating a patient with increased intracranial pressure, comprising administering to the patient a therapeutically effective amount of a PAR1/thrombin receptor antagonist for the purpose of: a) reducing brain damage, gliosis, gliotic scar formation, and/or neuronal death resulting from the increased intracranial pressure, and/or b) facilitating recovery from neurological deficit resulting from the increased intracranial pressure, thereby treating the patient with increased intracranial pressure.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIG. 1A is a graph showing average infarct volume in wild type versus PAR1−/− knockout mice.

FIG. 1B is a graph showing average infarct volume (based on intensity of wild type lesion) in wild type versus PAR1−/− knockout mice.

FIG. 2A is a diagram showing the location of a nitrocellulose filter implanted into the cerebral cortex of a wild type or PAR-/- knockout mouse subsequent to generation of a cortical scalpel wound.

FIG. 2B is a diagram of a photomicrograph showing GFAP staining of astrocytes at the surface of a nitrocellulose filter implanted into the cortex of a wild type mouse subsequent to generation of a scalpel wound in the cortex of the mouse.

FIG. 2C is a diagram of a photomicrograph showing GFAP staining of astrocytes at the surface of a nitrocellulose filter implanted into the cortex of a PAR1 knockout mouse subsequent to generation of a scalpel wound in the cortex of the mouse.

FIG. 3 is a graph showing that activation of PAR1 by TFLLR can stimulate astrocyte proliferation, as indicated by BrdU incorporation.

FIG. 4A is a graph showing the effects of TFLLR and ATP on free intracellular calcium in cultured astrocytes from wild type mice.

FIG. 4B is a graph showing the effects of TFLLR and ATP on free intracellular calcium in cultured astrocytes from PAR1−/− knockout mice.

FIG. 5A is a diagram showing the experimental arrangement for electrophysiological recording from hippocampal slices.

FIG. 5B is a graph showing a time course of current-response amplitude to NMDA during 3 U/ml thrombin application; the inset shows representative current responses from this same cell to pressure-ejected 1 mM NMDA and 100 μM glycine were recorded at −70 mV before and during perfusion with 3 U/ml thrombin (30 nM). 100 pM D-2-amino-5-phosphonovaleric acid (APV) blocked the response to NMDA/glycine.

FIG. 5C is a graph showing the mean time course (±SEM) of current responses to NMDA plus glycine in cells treated with 3 U/ml thrombin or 3 U/ml thrombin+100 ATU hirudin. The number of cells is indicated in parentheses. The mean response time course is also shown for control cells that were superfused with ACSF.

FIG. 5D is a graph showing that the mean potentiation (±SEM) of NMDA receptor responses by thrombin (open bar; −70 mV) was blocked by an irreversible serine protease inhibitor with high selectivity for thrombin over plasmin. 500 nM PPACK (˜15×thrombin concentration) was preincubated for 5-15 minutes with 30 nM thrombin before application to slices. Some slices were pretreated with 10 μM bisindolylmaleimide (BIS) for 10 minutes. *p<0.05 compared to thrombin potentiation; Kruskal-Wallis ANOVA, Dunn post hoc test. Open circles show results from individual neurons.

FIG. 5E is a graph showing a scatter plot of the NMDA response amplitude (upper panel) vs. fold-potentiation by thrombin and membrane resistance vs. fold-potentiation by thrombin (lower panel). There was no significant correlation between these parameters or the level of thrombin-induced potentiation (p<0.5). Data from rat and mouse were pooled.

### DETAILED DESCRIPTION OF THE INVENTION

The present invention is based on the discovery that PAR1, also known as the thrombin receptor, is a mediator of neuronal death associated with stroke, head trauma, and other neurodegenerative diseases, conditions, and injuries to the central nervous system. Applicants have found that entry of serine proteases (such as plasmin and thrombin) into the brain parenchyma as a result of a neurodegenerative disease, condition, or injury can activate the PAR1/thrombin receptors present on the cell surfaces of neurons and glia, thereby bringing about harmful consequences, such as reactive gliosis/glial scar formation, inhibition of neuronal repair/regeneration, and ultimately, neuronal death.

This discovery makes possible the treatment of various neurodegenerative and/or neurotoxic diseases, conditions, and injuries, i.e., using PAR1/thrombin receptor antagonists that partially or fully inhibit PAR1/thrombin receptor activity in neurons and glia. Administration of PAR1/thrombin receptor antagonists that inhibit PAR1/thrombin receptor activity on neurons and glia can be used to treat any neurodegenerative and/or neurotoxic disease, condition, or injury which results in PAR1/thrombin receptor-mediated neuronal death and/or reactive gliosis/gliotic scar formation. Such diseases, conditions, and injuries include, but are not limited to: stroke, head trauma or spinal cord trauma (e.g., penetrating trauma, such as that caused by a gunshot or knife wound; crush trauma, such as that caused by being run over by a motor vehicle; stretch and/or compression trauma, such as that caused by a whiplash-type injury to the spinal cord; or blunt trauma, such as that caused by being struck in the head or back with an object, e.g., a baseball bat), intracerebral bleeding (e.g., due to a brain aneurysm or ruptured arteriovenous malformation), subarachnoid hemorrhage, global ischemia associated with cardiovascular or pulmonary surgery, post-traumatic epilepsy or seizure, status epilepticus, idiopathic and chronic epilepsy, Alzheimer's disease, Parkinson's disease, an infection of the central nervous system (e.g., by a bacterium, virus, fungus, parasite, or prion), upper motor neuron disease, lower spinal motor neuron disease, amyotrophic lateral sclerosis, demyelinating diseases such as multiple sclerosis, NMDA receptor overactivation, and any other disorder of or injury to the central nervous system that involves neuronal death an/or reactive gliosis/glial scar formation as a result of or exacerbated by PAR1/thrombin receptor activation on the cell surface of neurons and/or glia.

The methods of the invention can also be used to treat a patient during or following a hypoxic episode. Hypoxia can lead to brain damage and gliosis/glial scar formation, due to reduced hemoglobin content, poisoning of hemoglobin (e.g. by carbon monoxide), poisoning of brain respiratory enzymes (e.g. by cyanide) resulting in failure to utilize oxygen, reduced oxygen tension in blood following cessation of breathing or obstruction of the respiratory tract above the tracheal bifurcation, arrest of respiration after disease or injury to the spinal cord or thorax, arrest of alveolar gas exchange due to inhalation of inert gases or drowning, lowered oxygen content at high altitude or failure of pressurized vehicle (e.g. aircraft) or inhalation of inert gases during anesthesia, or the inability to utilize oxygen due to deficiency of glucose.

For example, anemic hypoxia can lead to brain damage and gliosis/glial scar formation, including hypoxia due to reduced hemoglobin content or poisoning of hemoglobin by, for example, carbon monoxide. Histopathic hypoxia can lead to brain damage and gliosis/glial scar formation due to poisoning of brain respiratory enzymes resulting in failure to utilize oxygen. Anoxic hypoxia can lead to brain/spinal cord damage and gliosis/glial scar formation due to reduced oxygen tension in blood following cessation of breathing, obstruction of the respiratory tract above the tracheal bifurcation, or if respiration is arrested after disease or injury to the spinal cord or thorax, or arrest of alveolar gas exchange due to inhalation of inert gases or drowning. Hypoxic hypoxia can lead to brain/spinal cord damage and gliosis/glial scar formation due to lowered oxygen content at high altitude or in failure of pressurized vehicle (e.g. aircraft) or inhalation of inert gases during anesthesia. And hypoglycemic hypoxia can lead to brain damage and gliosis/glial scar formation as result of the brain/spinal cords inability to utilize oxygen due to deficiency of glucose.

PAR1/Thrombin Receptor Antagonists

The methods of the invention may be practiced with any PAR1/thrombin receptor antagonist currently known or later discovered. By “PAR1/thrombin receptor antagonist” or “PAR1 antagonist” or “thrombin receptor antagonist” is meant any compound, be it natural or synthetic, that partially or fully inhibits PAR1 activity. PAR1/thrombin receptor antagonists for use in the methods of the invention either partially or completely inhibit PAR1 activity. By “PAR1 activity” or “thrombin receptor activity” is meant the ability of PAR1/thrombin receptor to mediate neuronal death and/or glial cell proliferation/gliosis. Inhibition of PAR1/thrombin receptor activity decreases the degree of neuronal cell death and/or or gliosis in a subject with a neurodegenerative disease, condition, or injury in which PAR1 plays a role in mediating neuronal death and/or gliosis.

For example, the methods of the invention may be practiced using the PAR1/thrombin receptor antagonists described in U.S. Pat. No. 6,017,890 (to Hoekstra et al.: “Azole Peptidomimetics as Thrombin Receptor Antagonists”), which is herein incorporated by reference in its entirety, and is specifically incorporated by reference for its teachings of compounds that function as thrombin receptor antagonists (see, e.g., column 2, line 31, through end of column 3 and Examples 1-10).

The methods of the invention may also be practiced using the PAR1/thrombin receptor antagonists described in U.S. Pat. No. 5,446,131 (to Maraganore: “Throribin Receptor Antagonists”), which is herein incorporated by reference in its entirety, and is specifically incorporated by reference for its teachings of compounds that function as thrombin receptor antagonists (see, e.g., the Abstract and the claims).

The methods of the invention may also be practiced using the PAR1/thrombin receptor antagonists described in U.S. Pat. No. 5,866,681 (to Scarborough: “Thrombin Receptor Antagonists”), which is herein incorporated by reference in its entirety, and is specifically incorporated by reference for its teachings of compounds that function as thrombin receptor antagonists (see, e.g., the Abstract, the claims, and Examples 1-16).

The methods of the invention may also be practiced using the PAR1/thrombin receptor antagonists described in U.S. Pat. No. 5,759,994 (to Coughlin: “Recombinant Thrombin Receptor and Related Pharmaceuticals”), which is herein incorporated by reference in its entirety, and is specifically incorporated by reference for its teachings of compounds that function as thrombin receptor antagonists (see, e.g., Examples 5 and 6, and the claims).

The methods of the invention may also be practiced using the PAR1/thrombin receptor antagonists described in U.S. Pat. No. 5,798,248 (to Coughlin: “Recombinant Thrombin Receptor and Related Pharmaceuticals”), which is herein incorporated by reference in its entirety, and is specifically incorporated by reference for its teachings of compounds that function as thrombin receptor antagonists (see, e.g., Examples 5 and 6, and the claims).

The methods of the invention may also be practiced using the PAR1/thrombin receptor antagonists described in Bernatowicz et al. (“Development of Potent Thrombin Receptor Antagonists.” J. Med. Chem. 39:4879-4887, 1996), which is herein incorporated by reference in its entirety, and is specifically incorporated by reference for its teachings of compounds that function as thrombin receptor antagonists (see, e.g., Tables 1-8).

The methods of the invention may also be practiced using the PAR1/thrombin receptor antagonists described in Vassallo et al. (“Structure-Function Relationships in the Activation of Platelet Thrombin Receptors by Receptor-Derived Peptides.” J. Biol. Chem. 267:6081-6085, 1992), which is herein incorporated by reference in its entirety, and is specifically incorporated by reference for its teachings of compounds that function as thrombin receptor antagonists (see, e.g., Table 1).

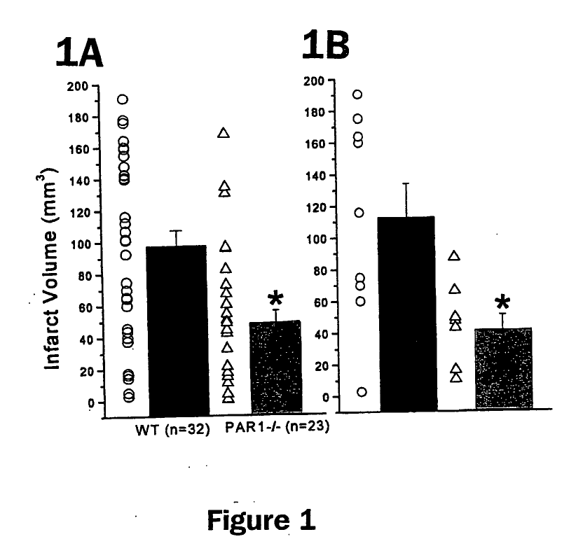

The methods of the invention may also be practiced using the PAR1/thrombin receptor antagonists described in Andrade-Gordon et al. (“Design, Synthesis, and Biological Characterization of a Peptide-Mimetic Antagonist for a Tethered-Ligand Receptor.” Proc. Natl. Acad. Sci. USA 96:12257-12262, 1999), which is herein incorporated by reference in its entirety, and is specifically incorporated by reference for its teachings of compounds that function as thrombin receptor antagonists (see, e.g., FIG. 1).

The methods of the invention may also be practiced using the PAR1/thrombin receptor antagonists described in Hoekstra et al. (“Thrombin Receptor (PAR-1) Antagonists. Heterocycle-Based Peptidomimetics of the SFLLR Agonist Motif.” Bioorg. Med. Chem. Lett. 8:1649-1654, 1998), which is herein incorporated by reference in its entirety, and is specifically incorporated by reference for its teachings of compounds that function as thrombin receptor antagonists (see, e.g., Tables 1 and 2).

The methods of the invention may also be practiced using the PAR1/thrombin receptor antagonists described in Kato et al. (“In Vitro Antiplatelet Profile of FR171113, a Novel Non-Peptide Thrombin Receptor Antagonist.” Euro. J. Pharmacol. 384:197-202, 1999), which is herein incorporated by reference in its entirety, and is specifically incorporated by reference for its teachings of compounds that function as thrombin receptor antagonists (see, e.g., FIG. 1).

The methods of the invention may also be practiced using the PAR1/thrombin receptor antagonists described in Ruda et al. (“Identification of Small Peptide Analogues Having Agonist and Antagonist Activity at the Platelet Thrombin Receptor.” Biochem. Pharmacol. 37:2417-2426, 1988), which is herein incorporated by reference in its entirety, and is specifically incorporated by reference for its teachings of compounds that function as thrombin receptor antagonists (see, e.g., the Abstract and FIG. 1).

The methods of the invention may also be practiced using the PAR1/thrombin receptor antagonists described in Ruda et al. (“Thrombin Receptor Antagonists: Structure-Activity Relationships for the Platelet Thrombin Receptor and Effects on Prostacyclin Synthesis by Human Umbilical Vein Endothelial Cells.” Biochem. Pharmacol. 39:373-381, 1990), which is herein incorporated by reference in its entirety, and is specifically incorporated by reference for its teachings of compounds that function as thrombin receptor antagonists (see, e.g., Table 2).

The methods of the invention may also be practiced using the PAR1/thrombin receptor antagonists described in Harmon and Jamieson (“Activation of Platelets by Alpha-Thrombin is a Receptor-Mediated Event.” J. Biol. Chem. 261:15928-15933, 1986), which is herein incorporated by reference in its entirety, and is specifically incorporated by reference for its teachings of compounds that function as thrombin receptor antagonists (see, e.g., the abstract at page 15928, left column).

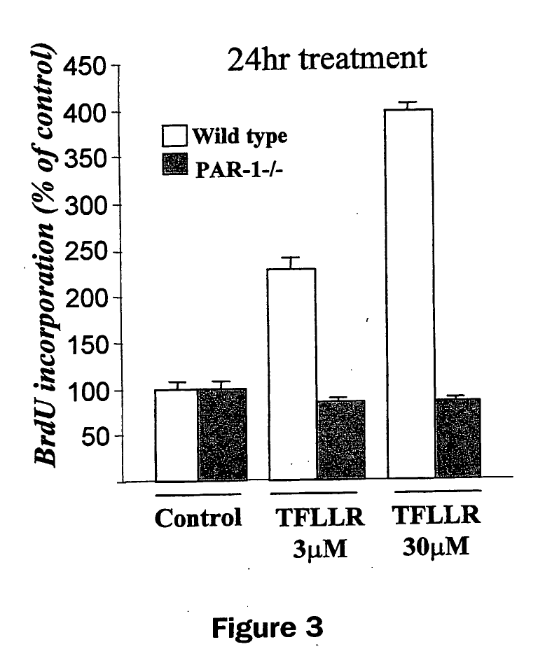

The methods of the invention may also be practiced using the PAR1/thrombin receptor antagonists described in Doorbar and Winter (“Isolation of a Peptide Antagonist to the Thrombin Receptor Using Phage Display.” J. Mol. Biol. 244:361-369, 1994), which is herein incorporated by reference in its entirety, and is specifically incorporated by reference for its teachings of compounds that function as thrombin receptor antagonists (see, e.g., FIG. 3).

The methods of the invention may also be practiced using the PAR1/thrombin receptor antagonists described in Ahn et al. (“Structure-Activity Relationships of Pyrroloquinazolines as Thrombin Receptor Antagonists.” Bioorg. Med. Chem. Lett. 9:2073-2078, 1999), which is herein incorporated by reference in its entirety, and is specifically incorporated by reference for its teachings of compounds that function as thrombin receptor antagonists (see, e.g., Tables 1 and 2). The methods of the invention may also be practiced using the PAR1/thrombin receptor antagonists described in Seiler et al. (“Inhibition of Thrombin and SFLLR-Peptide Stimulation of Platelet Aggregation, Phosphlipase A₂ and Na⁺/H⁺ Exchange by a Thrombin Receptor Antagonist.” Biochem. Pharmacol. 49:519-528, 1995), which is herein incorporated by reference in its entirety, and is specifically incorporated by reference for its teachings of compounds that function as thrombin receptor antagonists (see, e.g., the Abstract).

The methods of the invention may also be practiced using the PAR1/thrombin receptor antagonists described in Elliot et al. (“Photoactivatable Peptides Based on BMS-197525: A Potent Antagonist of the Human Thrombin Receptor (PAR-1).” Bioorg. Med. Chem. Lett. 9:279-284, 1999), which is herein incorporated by reference in its entirety, and is specifically incorporated by reference for its teachings of compounds that function as thrombin receptor antagonists (see, e.g., Table 1).

The methods of the invention may also be practiced using the PAR1/thrombin receptor antagonists described in Fujita et al. (“A Novel Molecular Design of Thrombin Receptor Antagonists.” Bioorg. Med. Chem. Lett. 9:1351-1356, 1999), which is herein incorporated by reference in its entirety, and is specifically incorporated by reference for its teachings of compounds that function as thrombin receptor antagonists (see, e.g., the Abstract).

The methods of the invention may also be practiced using the PAR1/thrombin receptor antagonists described in Debeir et al. (“Pharmacological Characterization of Protease-Activated Receptor (PAR-1) in Rat Astrocytes.” Euro. J. Pharmacol. 323:111-117, 1997), which is herein incorporated by reference in its entirety, and is specifically incorporated by reference for its teachings of compounds that function as thrombin receptor antagonists (see, e.g., the Abstract).

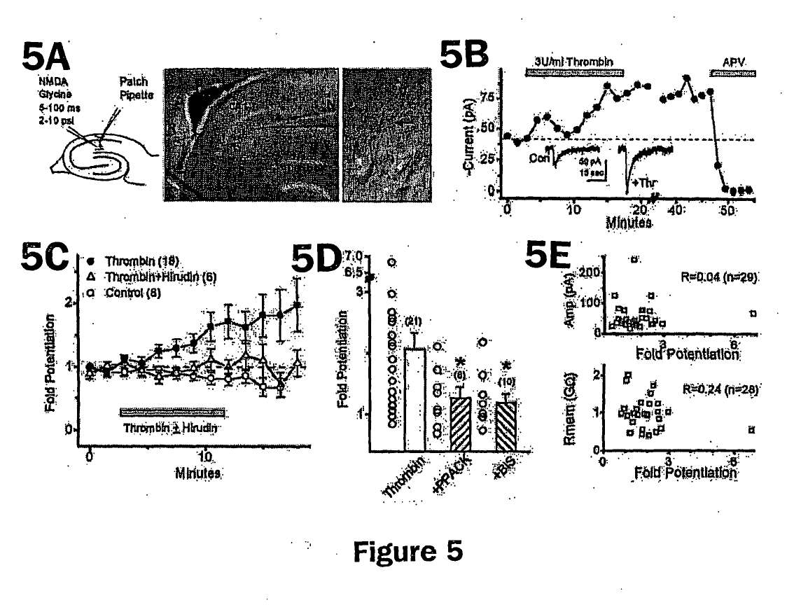

The methods of the invention may also be practiced using the PAR1/thrombin receptor antagonists described in Ahn et al. (“Binding of a Thrombin Receptor Tethered Ligand Analogue to Human Platelet Thrombin Receptor.” Mol. Pharmacol. 51:350-356, 1997), which is herein incorporated by reference in its entirety, and is specifically incorporated by reference for its teachings of compounds that function as thrombin receptor antagonists (see, e.g., FIG. 5 and Table 1).

The methods of the invention may also be practiced using the PAR1/thrombin receptor antagonists described in McComsey et al. (“Heterocycle-peptide hybrid compounds. Aminotriazole-containing agonists of the thrombin receptor (PAR-1).” Bioorganic & Medicinal Chemistry Letters 9: 1423-1428, 1999), which is herein incorporated by reference in its entirety, and is specifically incorporated by reference for its teachings of compounds that function as thrombin receptor antagonists (see, e.g., Table: Biological Data).

The methods of the invention may also be practiced using the PAR1/thrombin receptor antagonists described in Nantermet et al. (“Discovery of a small molecule antagonist of the human platelet thrombin receptor (PAR-1).” Bioorganic & Medicinal Chemistry Letters 12: 319-323, 2002), which is herein incorporated by reference in its entirety, and is specifically incorporated by reference for its teachings of compounds that function as thrombin receptor antagonists (see, e.g., Table 1, Table 2, Table 3).

The methods of the invention may also be practiced using the PAR1/thrombin receptor antagonists described in Barrow et al. (“Discovery and initial structure-activity relationship of trisubstituted ureas as thrombin receptor (PAR-1) antagonists.” Bioorganic & Medicinal Chemistry Letters 11: 2691-2696, 2001), which is herein incorporated by reference in its entirety, and is specifically incorporated by reference for its teachings of compounds that function as thrombin receptor antagonists (see, e.g., Table 1-5).

The methods of the invention may also be practiced using the PAR1/thrombin receptor antagonists described in Ahn et al. (“Inhibition of cellular action of thrombin by N3-cyclopropyl-7[[4-(1-methylethyl)phenyl]methyl]-7H-pyrrole[3,2f] quinazoline-1,3-diamine (SCH79797), a non-peptide thrombin receptor antagonist.” Biochemical Pharmacol 60: 1425-1434, 2000), which is herein incorporated by reference in its entirety, and is specifically incorporated by reference for its teachings of compounds that function as thrombin receptor antagonists (see, e.g., FIG. 1).

The methods of the invention may also be practiced using the PAR1/thrombin receptor antagonists described in Chackalamannil (“Thrombin receptor antagonists as novel therapeutic targets.” Curr Opin Drug Discovery Development 4: 417-427, 2001), which is herein incorporated by reference in its entirety, and is specifically incorporated by reference for its teachings of compounds that function as thrombin receptor antagonists.

The methods of the invention may also be practiced using the PAR1/thrombin receptor antagonists described in Stead et al. (“Eryloside F, a novel penasterol disaccharide possessing potent thrombin receptor antagonist activity.” Bioorg. Med. Chem. Lett. 10:661-664, 2000), which is herein incorporated by reference in its entirety, and is specifically incorporated by reference for its teachings of compounds that function as thrombin receptor antagonists (see, e.g., FIG. 1).

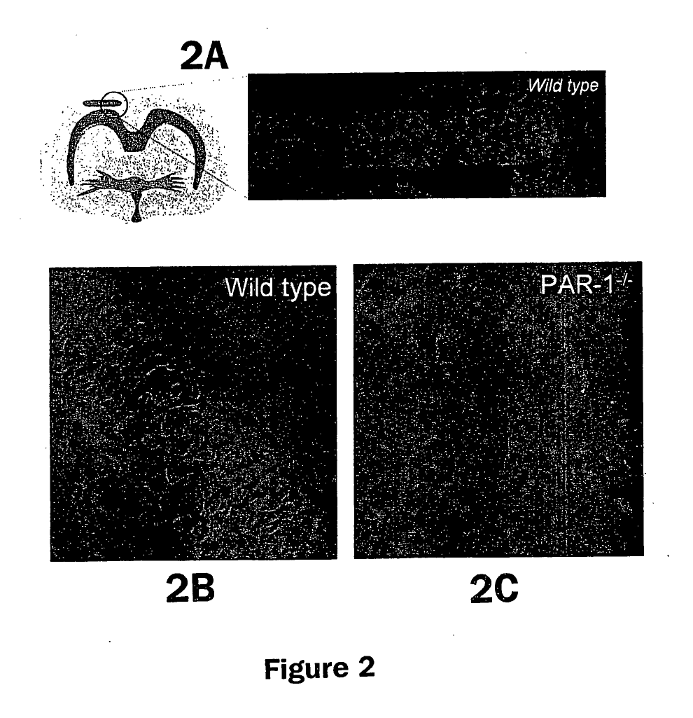

The methods of the invention may also be practiced using the PAR1/thrombin receptor antagonists described in Pakala et al. (“A peptide analogue of thrombin receptor-activating peptide inhibits thrombin and thrombin-receptor-activating peptide-induced vascular smooth muscle cell proliferation.” J. Cardiovasc. Pharmacol. 37: 619-629, 2001), which is herein incorporated by reference in its entirety, and is specifically incorporated by reference for its teachings of compounds that function as thrombin receptor antagonists (see, e.g., FIGS. 1 and 2).

The methods of the invention may also be practiced using the PAR1/thrombin receptor antagonists described in Zhang et al. (“Discovery and optimization of a novel series of thrombin receptor (PAR-1) antagonists: potent, selective peptide mimetics based on indole and indazole templates.” J. Med. Chem. 44: 1021-1024, 2001), which is herein incorporated by reference in its entirety, and is specifically incorporated by reference for its teachings of compounds that function as thrombin receptor antagonists.

Methods of Administration of PAR1/Thrombin Receptor Antagonists

The PAR1/thrombin receptor antagonists for use in the methods of the invention can be administered to subjects with a pharmaceutically acceptable diluent, carrier, or excipient, in unit dosage form. By “pharmaceutically acceptable” is meant a material that is not biologically or otherwise undesirable, i.e., the material may be administered to an individual along with a PAR1/thrombin receptor antagonist without causing any undesirable biological effects or interacting in a deleterious manner with any of the components of the pharmaceutical composition in which it is contained. Conventional pharmaceutical practice may be employed to provide suitable formulations or compositions to administer such compositions to subjects. Any appropriate route of administration may be employed, for example, but not limited to, intravenous, parenteral, transcutaneous, subcutaneous, intramuscular, intracranial, intraorbital, ophthalmic, intraventricular, intracapsular, intraspinal, intracistemal, intraperitoneal, intranasal, intrarectal, intravaginal, aerosol, or oral administration. Therapeutic formulations may be in the form of liquid solutions or suspensions; for oral administration, formulations may be in the form of tablets or capsules; for intranasal formulations, in the form of powders, nasal drops, or aerosols; for intravaginal formulations, vaginal creams, suppositories, or pessaries; for transdermal formulations, in the form of creams or distributed onto patches to be applied to the skin.

Methods well known in the art for making formulations are found in, for example, Remington: The Science and Practice of Pharmacy (19th ed.) ed. A. R. Gennaro, Mack Publishing Company, Easton, Pa. 1995. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for molecules of the invention include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Dosage

The PAR1/thrombin receptor antagonists for use in the methods of the invention may be administered to a subject in an amount sufficient to inhibit PAR1/thrombin receptor activity, in a subject in need thereof, e.g., to minimize damage to the nervous system (e.g., neuronal death and/or gliosis) in a subject in need of such treatment. One of ordinary skill in the art will understand that the optimal dosages used will vary according to the individual being treated and the particular neurodegenerative disease, condition, or injury for which the individual is being treated, the particular compound being used, and the chosen route of administration. The optimal dosage will also vary among individuals on the basis of age, size, weight, gender, and physical condition. Methods for determining optimal dosages are described, for example, in Remington: The Science and Practice of Pharmacy (19th ed.) ed. A. R. Gennaro, Mack Publishing Company, Easton, Pa. 1995. Typically, a pharmaceutically effective dosage would be between about 0.001 and 100 mg/kg body weight of the subject to be treated.

Efficacy

The efficacy of administration of a particular dose of a PAR1/thrombin receptor antagonist according to the methods of the invention can be determined by evaluating the particular aspects of the medical history, signs, symptoms, and objective laboratory tests that are known to be useful in evaluating the status of a subject in need inhibition of PAR1/thrombin receptor for the treatment and/or prophylaxis for a neurodegenerative disease, condition, or injury. These signs, symptoms, and objective laboratory tests will vary, depending upon the particular disease or condition being treated or prevented, as will be known to any clinician who treats such subjects or a researcher conducting experimentation in this field. For example, if, based on a comparison with an appropriate control group and knowledge of the normal progression of the disease or condition in the general population or the particular individual: 1) a subject's frequency or severity of recurrences is shown to be improved, 2) the progression of the neurodegenerative disease or condition is shown to be stabilized or delayed, 3) the damage to the central nervous system as a result of the disease, condition, or injury is less than that which would have occurred had the antagonist not been administered, or 4) the need for use of other medications for treating the neurodegenerative disease, condition, or injury is lessened or obviated, then a particular treatment will be considered efficacious. A therapeutically effective treatment will decrease the amount of damage to the central nervous system (e.g., neuronal death and/or gliosis) caused by the neurodegenerative disease, condition, or injury (relative to the amount of damage that would have occurred had the antagonist not been administered) by at least 10%, more preferably by at least 20%, 30%, 40%, 50%, 60%, 70%, 80%, or 90%, or greater.

### EXAMPLES

The present invention is more particularly described in the following examples, which are intended as illustrative only since numerous modifications and variations thereof will be apparent to those of ordinary skill in the art.

### Example I

### PAR1 Mediates Neuronal Damage Caused by Transient Focal Ischemia

This example shows that mice lacking protease activated receptor-1 (PAR1) have over a 2-fold reduction in infarct volume following focal transient ischemia. These findings indicate that PAR1 is one of the mediators of the neuropathological effects of serine proteases such as thrombin, tissue plasminogen activator (tPA) and plasmin during ischemia, and therefore provides a new therapeutic target for situations in which the blood-brain barrier is compromised.

Recent findings have implicated the tPA/plasmin system as a component of the signaling cascade resulting in neuronal death in certain pathological situations. Potential harmful effects of tPA are also relevant for stroke patients who are administered tPA intravenously as a thrombolytic agent to facilitate reperfusion of ischemic tissue. Since PAR1 can be activated by serine proteases involved in the blood clotting system, such as plasmin and thrombin, we sought to determine whether PAR1 plays a role in mediating the pathological consequences to neurons following ischemia using a murine stroke model of transient focal ischemia.

PAR1 is a seven transmembrane G-protein coupled receptor that stimulates G-alpha-q to initiate phosphatidyl inositol (PI) hydrolysis. To ensure that PAR1 is functional in regions of the brain that are subject to ischemic damage in the mouse transient focal ischemia model, we tested the ability of several activators of PAR1 to initiate PI hydrolysis (Chung et al., 1997) in the hippocampus and striatum. Both thrombin and plasmin increased PI hydrolysis in wild type hippocampus, but not in the hippocampus of PAR1−/− mice. Pre-incubation with a PAR1 antagonist peptide (N-trans-cinnamoyl-p-fluoroPhe-p-guanidinoPhe-Leu-Arg-Arg-NH₂) inhibited PI hydrolysis produced by both thrombin and plasmin. Furthermore, the specific PAR1 agonist peptide TFLLR-NH₂ (SEQ ID NO: 1) increased PI hydrolysis in hippopcampal slices from wild type but not from PAR1−/− knockout animals.

Since tPA activates plasminogen to form plasmin, we tested whether exogenously applied tPA could activate endogenous plasminogen in brain slices to form plasmin and activate PAR1. Application of tPA to wild type hippocampal slices significantly increased PI hydrolysis compared with application to PAR1−/− hippocampal slices. The plasmin inhibitor, alpha-2-antiplasmin, inhibited the tPA-induced increase in PI hydrolysis in wild type hippocampal slices; moreover, application of tPA did not increase PI hydrolysis in plasminogen-deficient mice. These results demonstrate that tPA is capable of activating endogenous plasminogen to form plasmin, which in turn activates PAR1 to increase PI hydrolysis.

Since our data indicate tPA is capable of forming plasmin to activate PAR1, we hypothesized that PAR1 could be a potential downstream substrate for the detrimental actions of the tPA/plasmin system. To test this hypothesis, we performed transient middle cerebral artery (MCA) occlusion surgeries (Kim et al., 2002; Gasche et al., 2001) in PAR1−/− and wild type mice, to mimic the effects of stroke, as follows.

Transient focal cerebral ischemia was induced by intraluminal middle cerebral artery (MCA) blockade with a nylon suture. The mice were anesthetized with 1.5% isoflurane in 30% oxygen and 70% nitrous oxide using a face mask. The rectal temperature was controlled at 37° C. with a homeothermic blanket. The mouse's femoral artery was cannulated to allow for the monitoring of blood pressure and arterial blood gases, and samples for analysis were taken immediately after cannulation, 10 min after occlusion, and 10 min after reperfusion. After a midline skin incision, the left external carotid artery was exposed, and its branches were electrocoagulated. An 11 mm monofilament nylon suture, blunted at the end, was introduced into the left internal carotid artery through the external carotid artery stump. After 30 min of MCA occlusion, blood flow was restored by the withdrawal of the nylon suture. After a 24 hour survival period, the brain was removed and sectioned into 2 mm sections. Occlusion of the left MCA for 30 minutes reduced blood flow by 80% as measured by laser Doppler flow to the left hemisphere, and reperfusion was then allowed for 24 hours. Mice were sacrificed 24 hours post surgery and brains immediately removed and coronally sectioned in 2 mm sections. Brain sections were incubated in the vital dye 2% triphenyltetrazolium chloride (TTC; Ito et al., 1997; Rich et al., 2001) The sites of interaction of triphenyltetrazolium chloride with mitochondrial respiratory chains. FEMS Microbiol. Lett. 21;202(2):181-7) in saline for 15-20 minutes at 37° C. The area of the lesion was measured using NIH image and the total infarct volume was determined by adding the individual section infarct volumes. A ratio of the contralateral to ipsilateral hemisphere section volume was multiplied by the corresponding infarct section volume to correct for edema.

The lesion in the wild type sections was clearly more severe than the lesion in the PAR1−/− sections. We took a conservative approach in determining the infarct volume such that we not only measured areas of the section that clearly showed neuronal degeneration as in the wild type, but also measured areas in which the neuronal degeneration was very slight, as in the PAR1−/− mice. Using this approach, PAR1−/− mice had a significant reduction (D=0.005) in total infarct volume (47.5+8.2 mm³) compared with wild type mice (97.3+9.7 mm³); (FIG. 1A; circles represent wild type infarct volumes and triangles represent PAR1−/− infarct volumes).

To directly compare the level of damage following MCA occlusion between the wild type and PAR1−/− mice, the grayscale scanned brain section images were segmented on the basis of intensity. The density slice option in NIH Image was used to segment the images on the basis of intensity. The intensity was set by selecting the most severely damaged area in a wild type lesion as the standard. This standard was maintained throughout the analysis and only objects at this intensity were highlighted and their area measured. Using the wild type lesion as the standard, the PAR1−/− mice showed a dramatic reduction compared to the wild type (FIG. 1B; circles represent wild type infarct volumes and triangles represent PAR1−/− infarct volumes). Specifically, average infarct volume based on intensity of wild type lesion was 112.6+21.4 mm³ (p=0.01) in wild type mice compared to 41.3+27.7 in PAR1−/− mice.

Average physiological parameters were measured before, during, and after surgery in subset of animals (n=4 wt, n=4 PAR1−/−) as follows. The femoral artery was cannulated and arterial blood pressure, blood gases, and pH were measured. Blood gases and pH were measured with a pH/Blood Gas Analyzer (Chiron Diagnostics Ltd.). Changes in regional cerebral blood flow were monitored with a laser Doppler flowmeter (LASERFLO BPM², Vasomedic). The laser Doppler probe was placed on the skull above the MCA territory cortex and measured before and after MCA occlusion. No differences between wild type and PAR1−/− mice were observed in physiological parameters (such as blood pH, CO2 pressure, O2 pressure, blood pressure, body temperature, and percent decrease in cerebral blood flow) monitored before, during, and after the surgery.

To ensure there were no obvious differences in brain anatomy as well as vasculature between the wild type and PAR1−/− mice that could account for the differential sensitivity to experimental ischemia, we compared the cerebral vasculature in wild type and PAR1−/− animals. Since most of the damage following MCA occlusion occurs in the striatum, hippocampus, and cortex, our analysis focused on these brain regions. Four wild type and four PAR1−/− mice were perfused with 2% carbon black in 20% gelatin in water to evaluate the major vessels. We observed no obvious differences in the major vessels between wt and PAR1−/− mice including the circle of Willis and the posterior communicating arteries.

In order to compare cellular morphology, brain anatomy, and vascular density, wild type and PAR1−/− standard histological brain section were analyzed both qualitatively and quantitatively. The PAR1−/− standard histological sections indicate that there are no apparent vascular malformations or structural abnormalities in the brain of animals lacking PAR1. In addition, the endothelial cells, capillaries, perivascular cells, and spaces appeared normal. Quantitatively, there were no differences between the wild type and PAR 1-/- vascular densities in the striatum, hippocampus, and cortex, suggesting there was no additional blood supply to these regions in the PAR1−/− mice that would account for the reduction in infarct volume.

Ultrastructural analysis of brain capillaries was completed to evaluate some of the components of the blood-brain barrier and to determine the average capillary lumen area. Although the standard histological sections revealed no evident differences in wild type and PAR1−/− mice at the cellular and anatomical levels, evaluating the blood brain barrier is vital since blood-brain barrier breakdown is a principal step in the pathological process during and after ischemia. Capillaries from the CA1 region of the hippocampus, the striatum and the cortex from five wild type and five PAR1−/− mice were analyzed for the presence of tight junctions, endothelial cell morphology, the degree to which astrocytic endfeet contacted the endothelial cells as determined by glial fibrillary acidic protein (GFAP) staining, and capillary lumen area.

Overall, there were no noticeable qualitative differences in wild type and PAR1−/− capillaries. Both wild type and PAR1−/− mice had one to two endothelial cells forming the capillary with a basal lamina of similar thickness surrounding them. The tight junctions between two endothelial cells or the same endothelial cell appeared similar and were observed at the same frequency in the wild type and PAR1−/− mice. GFAP staining indicates that PAR1−/− and wild type capillaries and vessels are surrounded by astrocytic endfeet. These results together suggest that the PAR 1-/- mice did not experience increased neuroprotection due to increased blood flow to the striatum, hippocampus, or cortex or due to alterations in their blood brain barrier.

Even though our data revealed there were no significant differences between the blood brain barriers of wild type and PAR1−/− mice, it's possible that during ischemia the blood brain barrier in PAR1−/− mice did not breakdown to the same degree as in wild type mice due to factors undetectable at the ultrastructural level. Since PAR1 is expressed on endothelial as well as smooth muscle cells and PAR1 activation has been shown to cause constriction of endothelial cells, we evaluated Evan's blue entry into brain tissue in ischemic and nonischemic conditions to test whether PAR1 played a role in blood-brain barrier breakdown. Evan's blue (2%) in saline was injected into the jugular vein of wild type (n=4) and PAR1−/− (n=4) mice immediately before the MCA occlusion surgery. Evan's blue is a 2 kD molecule that is normally restricted to the vascular space in the CNS but can cross the blood brain barrier during experimental ischemia. Mice that underwent a sham surgery and mice that underwent MCA occlusion for 30 minutes were sacrificed four hours post surgery to assess the amount of Evan's blue entry into the brain tissue. The four hour time point was chosen since previous studies have established that blood brain barrier breakdown does occur at this point following MCA occlusion. At longer time points multiple factors contribute to blood-brain barrier breakdown making it less likely that there would be an observable difference in Evan's blue entry into the brain tissue due to PAR1.

The results show that there were approximately equal amounts of Evan's blue entry into ipsilateral hemisphere of wild type and PAR1−/− mice, suggesting little difference in blood-brain barrier breakdown between the two groups. No Evan's blue fluorescence was observed in the contralateral hemispheres or in the nonischemic animals, indicating that the blood-brain barrier was intact and functional for both groups. Since lack of PAR1 does not appear to prevent blood brain barrier breakdown, the protective effects observed in PAR1−/− mice appear to occur at the neuronal level.

The results of this study indicate that a previously unrecognized signaling pathway in the central nervous system (CNS) mediates some of the pathological consequences of experimental ischemia. Specifically, lack of PAR1 confers significant neuroprotection following focal transient ischemia, suggesting PAR1 has an important role in mediating neurodegeneration in ischemic situations. Since we did not find any differences between the wild type and PAR1−/− mice in ischemic blood-brain barrier breakdown, PAR1 is likely to be initiating signaling cascades at the neuronal or glial level that contribute to cell death. As described herein, PAR1 activation has been shown to cause considerable potentiation of NMDA receptor responses and apoptosis in neurons, both of which contribute to the pathological process following experimental ischemia.

In recent years the tPA/plasmin sytem has been under increased scrutiny since tPA is currently used as thrombolytic therapy for stroke patients. As described above, tPA exogenously applied to hippocampal slices can convert endogenous plasminogen to plasmin, which activates PAR1. Extravasated plasmin has been shown to have numerous effects in the CNS, including cell death, short-term potentiation of synaptic transmission, decreased GABA inhibition and increased sensitivity to excitotoxins and NMDA. Our finding that lack of PAR1 confers significant neuroprotection suggests a potential mechanism for the pathological effects of tPA/plasmin system in that plasmin may activate PAR1 to cause neuronal damage. Moreover, our finding of an important role of PAR1 in cell death creates new opportunities for pharmacological intervention in stroke patients. For example, tPA thrombolytic therapy could be improved by first administering a therapeutic agent to inhibit PAR1 signaling in neurons and/or glia. Such therapy would inhibit the neurotoxic action of tPA, i.e., tPA-induced plasmin formation, but still allow tPA-stimulated reperfusion of brain tissue.

### REFERENCES

1. Chung et al. (1997) 4-Methylhomoibotenic acid activates a novel metabotropic glutamate receptor coupled to phosphoinositide hydrolysis. J. Pharmacol. Exp. Ther. 283: 742-9.

2. Kim et al. (2002) Manganese superoxide dismutase deficiency exacerbates cerebral infarction after focal cerebral ischemia/reperfusion in mice: implications for the production and role of superoxide radicals. Stroke. 33(3):809-15.

3. Gasche et al. (2001) Matrix metalloproteinase inhibition prevents oxidative stress-associated blood-brain barrier disruption after transient focal cerebral ischemia. J Cereb. Blood Flow Metab. 21(12): 1393-400.

4. Ito et al. (1997). Infarct size measurement by triphenyltetrazolium chloride staining versus in vivo injection of propidium iodide. J. Mol. Cell. Cardiol. 29(8):2169-75.

5. Rich et al. (2001) The sites of interaction of triphenyltetrazolium chloride with mitochondrial respiratory chains. FEMS Microbiol. Lett. 21;202(2):181-7.

### Example II

### PAR1 Stimulates Astrocyte Proliferation and is Necessary for Gliotic Scar Formation in the Central Nervous System

All central nervous system (CNS) injury induces both the migration of microglia to the site of injury as well as an increase in the number of glial-fibrillary acidic protein (GFAP)-expressing astrocytes. This reactive gliosis is a characteristic feature of glial scarring. Moreover, the gliotic scar has been considered to be an impediment to neuronal repair. Gliotic scars and biochemical changes in the extracellular environment produced by reactive astrocytes are thought to form a barrier that inhibits axon growth and neuronal repair, thus hindering the re-establishment of appropriate neuronal connections in the injured area. This barrier is hypothesized to be a cause of prolonged and often-incomplete recovery of function in affected regions; however, because there has been no known way to eliminate gliotic scar formation, this hypothesis has never been tested.

We have discovered that activation of the protease activated receptor-1 (PAR1) in vivo is a required step in the onset of reactive gliosis, as measured by up-regulation of GFAP in a model of penetrating head injury. As evidence of this discovery is our observation that mice lacking the PAR1 receptor do not show any evidence of gliotic scar formation in response to scalpel wound in the cortex, when compared at 7 days post injury to wild type mice (n=3 pairs of animals). This is evidenced by immunostaining for glial acidic fibrillary protein (GFAP), a hallmark for reactive astrocytes. FIGS. 2A-2C show a comparison of the astrocytic reaction at the surface of a nitrocellulose filter, implanted after generating a scalpel wound of the cortex, between wild type and PAR1−/− mice. Immunohistochemical staining for GFAP was performed in the brain sections seven days after the scalpel wound and nitrocellulose filter implantation. High levels of GFAP reactivity were seen in astrocytes at the surface of the filter and in astrocytes extending radially away from the filter in wild type mice (FIG. 2B). Little or no reactivity was observed in PAR1−/− mice (FIG. 2C).

The above-described in vivo data are further supported by our demonstration that glial cells in vitro can proliferate in response to selective activation of PAR1, as evidenced by bromo-deoxyuridine (BrdU) incorporation. Cortical astrocytes from wild type and PAR1−/− knockout mice were either untreated (control) or treated for 24 hours with a 3 or 30 micromolar concentration of the peptide TFLLR-NH₂ (SEQ ID NO: 1), which selectively activates PAR1. Cells were pulse-labeled with BrdU for the final 2 hours of culture. Immunostaining showed that all BrdU-labeled cells were also positive for GFAP, indicating that the BrdU-labeled cells were astrocytes. Treatment with TFLLR-NH₂ increases the number of BrdU-positive cells in cultures from wild type mice, but not in cultures from PAR1−/− mice, indicating that astrocyte proliferation can be stimulated by PAR1 activation (FIG. 3). This PAR1-induced astrocyte proliferation could be inhibited by a 24-hour pre-treatment with pertussis toxin, an inhibitor of GᵢGₒ protein, as determined by cell counting of BrdU-positive cells.

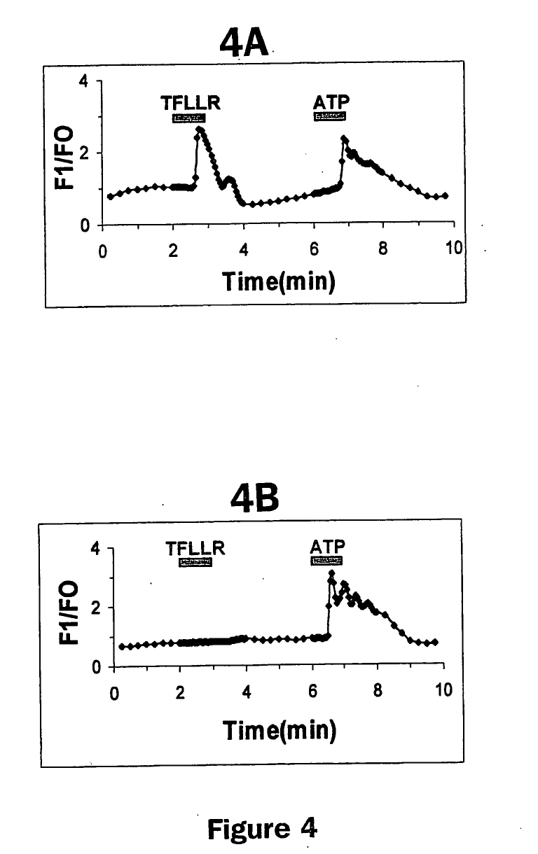

Further evidence that the peptide TFLLR-NH₂ acts on astrocytes in a PAR1-specific manner is demonstrated by the observation that TFLLR-NH₂ increases free intracellular calcium (as measured by the fluorescent calcium dye Fluo-3 AM) in cultured astrocytes from wild type mice (FIG. 4A), and does not increase free intracellular calcium in cultured astrocytes from PAR-/- knockout mice (FIG. 4B). By contrast, ATP increases free intracellular calcium in astrocytes from both wild type and PAR-/- mice, showing that the effects of TFLLR-NH₂ on astroyctes are PAR1-specific (FIGS. 4A-4B).

Our data demonstrate a required role for PAR1 activation in the gliotic response to penetrating head wound. From these data we predict that PAR1 activation will also be necessary for the gliotic response to traumatic brain injury, ischemic brain injury, brain hemorrhage, and spinal cord trauma. We further predict that PAR1 activation will influence the regulation of biomolecules that act as inhibitors to axon outgrowth and neuronal repair. Our finding creates an opportunity for therapeutic intervention that had not previously been known, as there is no known treatment that can reduce the formation of a gliotic scar. Our discovery of the critical role of PAR1 activation in gliotic scar formation indicates that antagonists that can block PAR1 activation can be used for the therapeutic prevention of gliotic scar formation in the central nervous system. Prevention of such gliotic scars will facilitate behavioral and cognitive recovery for patients suffering a wide range of central nervous system insults, including, but not limited to, hemorrhagic or occlusive stroke, head trauma, neck trauma, or spinal trauma, penetrating head wound or spinal cord injury, and/or central nervous system infection.

### Example III

### PAR1 Mediates Neuronal Damage Caused by Traumatic Brain Injury

In the study described below, we asked whether activation of PAR1 could influence the neuronal damage associated with traumatic brain injury using a rodent model of cortical and subcortical compression. Resulting injuries in this model involve a wide range of biochemical pathways, some of which are shared with ischemic injury. Furthermore, intra-cerebral hemorrhage is a component of such injury, one consequence of which is the introduction into brain parenchyma of blood-derived serine proteases such as thrombin, tissue plasminogen activator, Factor Xa, and the zymogen precursor of plasmin, plasminogen. Because Factor Xa, thrombin, and plasmin can activate PAR1, we tested whether PAR1 activation might mediate a portion of the neuronal death subsequent to injury.

A. Materials and Methods

Using aseptic procedures, unilateral contusions of the lateral frontoparietal cortex were induced using a custom-built controlled cortical impact (CCI) device. Male C57BL/6J wild type or PAR1−/− mice were anesthetized with isofluorane (5% induction; 3% maintenance) in 30/70% oxygen/nitrous oxide mixture. After securing the mouse in a stereotaxic frame, a 4-mm diameter craniotomy was performed over the left frontoparietal cortex with a standard tissue biopsy punch (center approximately −1.0 AP and +2.0 ML relative to bregma). The injury was produced by pneumatically activating a piston (tip diameter=3 mm), positioned at 15° from vertical in the coronal plane to achieve perpendicular impact of the cortical tissue, to a depth of 1 mm at a velocity of 6 m/s for a duration of 150 ms (Scherbel et al., Differential acute and chronic responses of tumor necrosis factor-deficient mice to experimental brain injury. Proc. Natl. Acad. Sci. USA 96: 8721-8726, 1999). Duration and velocity were verified in real-time via LVDT (Macrosensors, Pennsauken, N.J.) measurements. Following the procedure, the wound cavity was cleaned thoroughly and all bleeding stopped before suturing the scalp closed. At 7 days post injury animals were anesthetized, perfused with 4% paraformaldehyde, and the brain removed for histological analysis. Brains were cut in consecutive 16 micron sections, mounted on glass slides, thionin stained and digitally photographed (Media Cybernetics camera). The area of the ipsilateral and contralateral hippocampal formation was measured using ImagePro software, and hippocampal atrophy determined as the ratio of the ipsilateral to contralateral area.

B. Results

Analysis of the hippocampal area for wild-type mice revealed a profound atrophy that reflects neuronal loss at 7 days post-injury. The area of the ipsilateral hippocampus was reduced to 54±11% (mean+/−SEM, n=3 wild-type animals) of that determined from the contralateral hippocampus from the same animal. In contrast, PAR1−/− animals showed virtually no hippocampal atrophy at 7 days post injury. On average the area of the ipsilateral hippocampus was 95% (n=2 PAR1−/− animals) of that determined from the contralateral hippocampus. These data demonstrate a role for PAR1 in traumatic brain injury. These data also indicate that selective block of protease receptors in brain tissue will reduce the infarct associated with a traumatic brain and spinal cord injury. Reduction in the infarct should improve recovery and reduce loss of function for patients suffering head or spinal trauma.

### Example IV

### PAR1 Potentiation of NMDA Receptor Function

In the study described below, we asked whether the actions of thrombin on PAR1 could modify NMDA receptor function, which is an important contributor to both seizure initiation and neurodegeneration subsequent to cerebrovascular insult (e.g., ischemia) or traumatic brain/spinal cord injury. We observed that activation of PAR1 by thrombin potentiates hippocampal NMDA receptor responses, and the effects of thrombin can be mimicked by a peptide agonist (SFLLRN) that activates PAR 1. Morever, potentiation of the NMDA receptor by thrombin in hippocampal neurons is significantly attenuated in mice lacking PAR1. These data suggest that potentiation of neuronal NMDA receptor function after entry of thrombin, plasmin, or other serine proteases into brain parenchyma during intracerebral hemorrhage or extravasation of blood plasma proteins during blood-brain barrier breakdown can exacerbate glutamate-mediated cell death and participate in post-traumatic seizure.

A. Materials and Methods

Electrophysiological recording from rat and mouse hippocampal slices. Mice or rats (postnatal day 12-21; P12-P21) were anesthetized using isoflurane, decapitated, and the hippocampus rapidly dissected. Transverse hippocampal slices (250-300 microns) were cut in ice-cold artificial cerebrospinal fluid (ACSF) using a vibratome, and secured in a submerged recording chamber perfused with 1 micromolar tetrodotoxin and 10 micromolar bicuculline in ACSF. ACSF was composed of (in mM) 124 NaCl, 26 NaHCO₃, 2.5 KCl, 1 CaCl₂, 1.4 MgCl₂, 1 NaH₂PO₄, 10 glucose, and was saturated with 95% O₂-5% CO₂ (pH 7.4). In some experiments the extracellular recording solution was supplemented with 10 micromolar nifepidine (in 0.2% DMSO) to reduce Ca²⁺ currents. Blind and visually-guided whole cell patch recordings were obtained at 23° C. from CA1 pyramidal neurons using thin-walled 2.8-5.5 MOhm glass pipettes filled with a solution comprised of (in mM) 110 Cs-gluconate, 40 HEPES, 5 MgCl₂, 2 Na-ATP, 0.6 EGTA, 0.3 Na-GTP, with the pH adjusted to 7.3 using CsOH; osmolality was 275-290 MOsm. In some experiments, EGTA was omitted, 40 mM HEPES was replaced with 5 mM HEPES plus 30 mM CsCl, and the solution supplemented with 1 mM QX-314 (Sigma, St. Louis Mo.); similar results were obtained with both internal solutions. The presence of intracellular Cs⁺ should block GABA_{B} receptor-mediated currents. Brief (<100 msec) pulses of NMDA (0.3-2 mM) plus glycine (0.1-0.3 mM) were applied via pressurized pipette placed either in or just above stratum radiatum; the pressurized pipette was positioned to apply drug to the proximal third of the CA1 pyramidal cell dendrite; dilution at the tip was minimized prior to recording, and the tip was checked for blockage at the end of the experiment.

NMDA-evoked currents were recorded at −70 mV (corrected for the +10 mV measured junction potential) before, during, and after thrombin application. In some experiments membrane potential was changed to −40 mV during alternate agonist applications, or briefly jumped to −70 and −40 mV from a holding potential of 0 mV before and during agonist application. Series resistance (mean 23.4+2.3 MOhm) was monitored from the instantaneous current response to a −5 mV jump applied prior to agonist application, and the membrane resistance (mean 1.4+0.2 GOhm) was estimated from the leak current at −70 mV assuming a reversal potential of 0 mV. Series resistance compensation was not employed because the mean response amplitude (−49 pA) will cause only a 1 mV error in the holding potential and the slow response time course eliminates the capacitative component of series resistance filtering. Experiments with substantial changes in membrane or series resistance, regenerative currents, or development of leak currents exceeding −200 pA at −70 mV were excluded from analysis.

After 3-10 stable baseline measurements were taken, 3 U/ml alpha-thrombin (hereafter, thrombin; Calbiochem, La Jolla Calif.; Sigma, St. Louis Mo.; Haematological Technologies Inc., Essex Junction, Vt.) was applied through the bath solution for 10-18 minutes. In control experiments, ACSF was applied through the same perfusion line as thrombin. The perfusion line and recording chamber were washed extensively following experiments involving thrombin treatment since low pM levels of thrombin are capable of inactivating PAR receptors prior to recording. The specific activity of the thrombin from various vendors ranged between 1720-3200 NIH U/mg by comparison to Lot J of the NIH standard. In order to estimate the concentration of active thrombin that corresponds to 1 U/ml activity, we calculated a conversion factor using our most pure thrombin (3200 U/mg). Because the protein in this lot was reported by the manufacturer to be >95% thrombin as determined by gel electrophoresis, a solution with 1 U/ml thrombin should be 9 nM using a molecular weight for thrombin of 36.7 kDa. For simplicity, we used a conversion factor of 1 U/ml equals 10 nM thrombin throughout the text to estimate the concentration of active thrombin from various vendors. The peak potentiation of NMDA responses by thrombin was calculated over 20 minutes following thrombin application as the ratio of the highest running average of three consecutive response amplitudes (excluding the first 3 minutes after thrombin application) to the average of the three responses surrounding the time of thrombin application. Peak potentiation was used as a measure of thrombin's actions to remove the variability associated with the time required for thrombin to reach cells at different depths in the slice.

Imaging of Fluo-3 Fluorescence From Cultured Hippocampal Neurons.

Embryonic day (E)17-E19 rat pups were taken from CO₂-asphyxiated pregnant rats and the hippocampus dissected. Neurons were dissociated by trituration through a sterile fire-polished Pasteur pipette, centrifuged, and plated in Neurobasal (Life Technologies, Gaithersburg, Md.) defined media at a density of 60,000/ml on polylysine-coated (10 micrograms/ml) 12 mm glass coverslips. Media was supplemented with B27 nutrients (Gibco) plus penicillin/streptomycin, and cells were maintained 3-5 days at 37° C. in humidified 5% CO₂. Neurons were incubated for 30-45 minutes in a solution comprised of (in mM) 150 NaCl, 3 KCl, 10 HEPES, 2 CaCl₂, 20 mannitol, 10 glucose supplemented with 0.1% pluronic acid, 0.5% DMSO and 31M Fluo-3 acetoxymethyl ester (Molecular Probes, Eugene Oreg.); dye-loaded cells were placed in an identical solution lacking DMSO, pluronic acid, and Fluo-3. Images were acquired every 15 sec following 1 sec exposure to 450-490 nm light and fluorescence recorded through a bandpassed filter (500-550 nm) using a Photometrics CC200 CCD camera. 3 nM thrombin (0.3 U/ml)±50 nM D-Phe-Pro-Arg-chloromethylketone (PPACK; an irreversible thrombin inhibitor; Tapparelli et al., Synthetic low-molecular weight thrombin inhibitors: molecular design and pharmacological profile. Trends in Pharmacol. Sci. 14:366-376, 1993), as well as 10 μM PAR agonist peptide SFLLRN were applied in the presence of 0.5 μM tetrodotoxin (TTX) and 50 μM APV. 50 μM NMDA and 10 μM glycine were subsequently applied in the presence of 0.5 μM TTX. Fluorescence intensity was measured in cell bodies using image analysis software (Scion Corp., Frederick, Md.) and expressed as F/F_{O} where Fₒ is the fluorescence intensity prior to drug treatment. Increases in fluorescence greater than 1.2-fold were considered to be real changes since untreated cells possessed a peak F/Fₒ ratio of 1.1+0.04 over a typical experiment.

Transmitted Light Measurements in Hippocampal Slices.

Rat hippocampal slices were prepared as described above, and were bathed in a submerged chamber with 0.5 micromolar TTX, and 3-7 U/ml thrombin in 0.5 micromolar TTX applied for 10-20 minutes. The intensity of transmitted light (450-490 nm) through hippocampal slices was monitored as an indication of extracellular volume fraction (Andrew and MacVicar, Imaging cell volume changes and neuronal excitation in the hippocampal slice. Neuroscience 62: 371-383, 1994). Images were recorded using a Princeton Micromax CCD camera (Trenton, N.J.; 0.2 sec exposure every 20 to 40 sec) and analyzed using Axon Imaging Workbench 2.1 software (Foster City, Calif.). Intensity was expressed as I/Iₒ where Iₒ is the intensity of transmitted light prior to treatment. Solutions were made hyperosmotic by addition of 30 mM mannitol, which can produce a 10% expansion of the extracellular volume fraction (McBain et al. Regional variation of extracellular space in hippocampus. Science 249: 674-677, 1990). Hypoosmotic solutions were obtained by addition of 10% v/v water.

Genotyping of PAR1−/− mice. Male PAR1+/− mice were obtained from UCSF (gift from Dr. Shaun Coughlin), and have been described elsewhere (Connolly et al. Role of the thrombin receptor in development and evidence for a second receptor. Nature 381: 516-519, 1996). PAR1+/− (C57B1/6 background) mice were bred with female C57B1/6 wild-type mice from Jackson Laboratories (Bar Harbor, Me.), and the presence of PAR1 or neomycin gene determined using polymerase chain reaction (PCR) of genomic tail DNA obtained by digesting tails in 0.7 ml of a solution containing 0.1 M EDTA, 50 mM Tris, 0.5% SDS, 1 mg/ml proteinase K (pH 8.7) at 5055° C. for 4-5 hours; the digest was subsequently extracted with phenol/chloroform. The DNA was precipitated and re-suspended at 1 microgram/microliter in Tris/EDTA (10 mM/1 mM). PCR reactions were run with 1 microgram genomic DNA using the following protocols repeated through 40-45 cycles: 95° C. (30 sec), 85° C. (5 sec), 60° C. (2 min), 72° C. (2 min) for PAR1+/+ mice, and 95° C. (30 sec), 85° C. (5 sec), 58.5° C. (45 sec), 72° C. (1 min) for the neomycin resistance gene. The primers for PAR1 have been described (Connolly et al., 1996, supra); for neomycin the primer pair GAAGGGACTTGCTATTGG (SEQ ID NO: 2), GCTCTTCAGCAATATCACGGG (SEQ ID NO: 3) was used, which generates a 431 bp fragment. PCR reactions testing the genotype of unknown animals were carried out in parallel with control DNA from PAR1+/+, PAR1+/−, and PAR1−/− mice. Mice used in this study were derived from PAR1−/− breeding pairs (11), PAR1+/+ breeding pairs (4), or Jackson Laboratories wild-type mice (11). PCR results were repeated three times for all mice included in the study.

Expression and recording of NMDA and PAR1 receptor function in Xenopus laevis oocytes. cRNA was synthesized from linearized template cDNA according to manufacturer specifications (Ambion, Austin, Tex.). Xenopus oocytes were removed from ovaries of female frogs anesthetized with 0.3% 3-aminobenzoic acid ethylester. Groups of 20-30 stage V-VI oocytes were incubated in 292 U/ml Worthington (Freehold, N.J.) Type IV collagenase for 2 hours with slow shaking. The oocytes were rinsed in Barth's solution, and stored at 17° C. in Barth's solution supplemented with 100 microgram/ml gentamycin and 40 microgram/ml streptomycin. Control oocytes were injected with 3 ng NR1 subunit and 7 ng NR2B subunit. Other oocytes were injected with the NMDA receptor subunits plus 3 ng PAR1 cRNA. Two electrode voltage clamp recordings were made 3-7 days post-injection. Oocytes were continually perfused with recording solution (in mM, 90 NaCl, 1 KCl, 10 HEPES, pH 7.4) and held under voltage clamp at −30 to −50 mV with an OC-725B amplifier (Warner Instruments, Hamden, Conn.). Recording solution was supplemented with 1.0 mM CaCl₂ during initial impalement of the oocytes and subsequently 0.5 mM BaCl₂ during recording to reduce the calcium-activated chloride current endogenous to oocytes. Voltage and current electrodes had a resistance of 3-6 MOhms when filled with 0.3-3 M KCl. NMDA receptor currents were evoked by 20 micromolar glutamate/10 micromolar glycine perfused for 0.5-1 minute. PAR1 receptor activation was elicited by either 2.5 U/ml thrombin (Calbiochem, La Jolla, Calif.) or 10-30 micromolar agonist peptide SFLLRN (Bachem, Torrance, Calif.) for 2-3 minutes; the recording chamber and perfusion lines were washed extensively between thrombin applications since low pM levels of thrombin are capable of inactivating PAR receptors prior to recording (Vu et al., Molecular cloning of a functional thrombin receptor reveals a novel proteolytic mechanism of receptor activation. Cell 64: 1057-1068, 1991). Expression of PAR1 protein was verified by a thrombin or SFLLRN-stimulated rapidly desensitizing inward current at −30 mV that reflects the Ca²⁺-activated C1-current endogenous to the oocyte. To test the ability of thrombin to modify NMDA receptor function in oocytes, recordings of receptor function were made both before and after a 15-60 min incubation in recording solution supplemented with thrombin in Eppendorf tubes at room temperature. Only oocytes that showed less than 10% change in current between a pair of responses within each 10 min recording period were included in the analysis, in order to eliminate possible rundown of response. Oocytes with responses less than 50 nA were not included in the analysis.

NMDA receptor subunit immunoblots. Human embryonic kidney (HEK) cells were maintained in DMEM supplemented with 0.5 mM glutamine, 1 mM pyruvate, penicillin, streptomycin, and 10% fetal bovine serum in a humidified environment with 5% CO₂, and transfected with 1 microgram/ml of NR1 or 1:2 microgram/ml NR1:NR2 cDNA. Cells were washed in ice-cold HEPES buffered saline (HBS), scraped on ice, and membranes isolated by centrifugation at 12,000×g. N-terminal myc-tagged NR1-1a was a gift from Dr. R. Huganir (Johns Hopkins University, Baltimore Md.), and was constructed by inserting a myc tag (MEQKLISEEDLN; SEQ ID NO: 4) after Asn50, 29 residues after the signal peptide. Adult rat brain regions were dissected, frozen in liquid nitrogen, homogenized in ice-cold HEPES buffered saline (HBS), centrifuged, and stored at −20° C. Membranes were resuspended in HBS and treated with thrombin or buffer for 30 min at 37° C. Samples were centrifuged, and the membrane pellet resuspended in 2% sodium dodecylsulphate (SDS), 62.5 mM Tris, 10% glycerol, 5% beta-mercaptoethanol, 0.05% bromophenol blue (pH 6.8); some samples were treated with 10U DNase for 10 min at 37° C. SDS/polyacrylamide gel electrophoresis (SDS/PAGE) and protein blotting to Immobilon-P membranes was performed as described (Lau and Huganir, Differential tyrosine phosphorylation of N-methyl-D-aspartate receptor subunits. J Biol Chem 270: 20036-20041, 1995). Membranes were blocked for 30 min in 0.2 M Tris base, 1.37 M NaCl, pH 7.4, containing 5% nonfat dry milk, and incubated overnight at 4° C. in primary antibodies: NR1 mAb54.1 (gift from Dr. S. Heinemann, Salk Institute, La Jolla Calif.), NR1 alt-C-terminal (Upstate Biotechnology, Lake Placid, N.Y.), NR2A C-terminal (Chemicon, Temecula, Calif.), or NR2B C-terminal (Upstate Biotechnology). Membranes were washed 3×, and incubated with HRP-conjugated goat anti-mouse or goat anti-rabbit antibodies (1:10,000), washed 3×, and developed using enhanced chemiluminescence. The percent of receptor cleaved by thrombin was determined using densitometry.

B. Results

Thrombin potentiation of NMDA receptor currents in rat hippocampal neurons Messenger RNA encoding the thrombin receptor PAR1, which is known to activate G_{Q}- and Gᵢ-linked intracellular signaling systems, is expressed in hippocampal neurons including CA1 pyramidal cells. We have studied the effects of thrombin on whole cell voltage clamp recordings of rat CA1 hippocampal pyramidal neuron responses to pressure application of NMDA plus glycine into the dendritic field to test whether serine proteases can alter NMDA receptor function through PAR1 activation.

FIG. 5A illustrates our experimental recording arrangement. The photomicrographs show typical placement of the pressurized pipette (N) in relation to recording pipette (R) in stratum pyramidale. The typical placement of the NMDA containing pipette (black arrow) is shown in relation to a single CA1 pyramidal cell (white arrow). The black arrowhead shows the CA1/CA3 boundary NMDA receptor current responses were evoked in the presence of 1 micromolar tetrodotoxin and 10 micromolar bicuculline. Typical NMDA-evoked current responses recorded at −70 mV are shown as a time course before and during the application of 30 nM thrombin (3 U/ml) for a representative cell (FIG. 5B). In this cell thrombin treatment potentiated the current response with a time course consistent with thrombin diffusion into the tissue.

To confirm that the enhanced inward current response arises from activation of NMDA receptors rather than strychnine-sensitive glycine receptors or sensitization of stretch-activated channels that might respond to pressure ejection of agonist, responses to NMDA/glycine were blocked by 100 micromolar of the competitive NMDA receptor antagonist APV (n=7 cells). Thrombin potentiation of NMDA responses in neurons with the pressurized pipette positioned <100 microns above the slice ruled out the possibility that thrombin sensitized the tissue to pressure. Thrombin produced on average a 2.07±0.27-fold peak potentiation (mean±SEM; n=21) of NMDA responses at −70 mV within 20 minutes compared to cells in which thrombin was not applied. Although potentiation was often rapid (within a few minutes), in some cells, thrombin potentiation continued to increase with time, reaching peak levels between 5- and 8-fold. The thrombin-induced peak potentiation of NMDA responses appeared to possess a bimodal distribution in which a subset of the cells showed little response to thrombin (FIG. 5D). This differential effect of thrombin is consistent with the expression of PAR1 mRNA observed in some but not all CA1 pyramidal cells.

On average there were no significant changes in series resistance (111+7%; p<0.2; paired t-test) during thrombin application. Average membrane resistance (1.2+0.1 MOhm) decreased modestly throughout the course of the experiment, however this decrease was not significantly different between thrombin-treated (12+6%) and control slices (16+7%; p<0.2; t-test). In addition, there was no significant correlation (p<0.5 in all cases) between the levels of thrombin potentiation and the following parameters: the series resistance (r=0.37), the change in series resistance (r=0.00), the membrane resistance (r=0.24; FIG. 5E), the change in membrane resistance (r=0.18), or the response amplitude (r=0.04; FIG. 5E).

Current responses recorded under voltage clamp to pressure application of NMDA reversed sign at +0.1 mV (n=6). Together these results suggest that the thrombin-induced potentiation of NMDA receptor function we observe does not reflect a thrombin-induced compromise of the voltage clamp. Indeed, an identical degree of thrombin potentiation of the NMDA response (1.8±0.2-fold) was observed in cells not included in FIG. 5D because either their input resistance was too low or there was an increase in series resistance throughout the experiment (n=12).

Furthermore, thrombin-induced potentiation was observed when cells were held at 0 mV to inactivate Ca²⁺ channels and the holding potential briefly stepped to −70 mV before and during agonist application (2.7+0.7-fold potentiation; n=7). There was no significant difference between the degree of potentiation seen under normal conditions or in cells held at 0 mV (p>0.05; Mann-Whitney test). These results argue against the possibility that asynchronous activation of PAR1/PKC-potentiated Ca²⁺ channels during NMDA-induced loss of voltage clamp in distal dendrites could account for the potentiation we observe.

To confirm that the potentiating effects of thrombin that we observe reflect the proteolytic actions of thrombin rather than non-specific effects or the actions of other contaminant proteases, we evaluated whether the potentiation of NMDA receptor responses by thrombin could be blocked by two selective thrombin antagonists. We first examined whether hirudin (Calbiochem, La Jolla, Calif.), an inhibitor that binds with high affinity to thrombin's anion binding exosite (K_{D}20fM), could alter the time course of thrombin potentiation of NMDA receptor function.

Hirudin significantly reduced the potentiation by thrombin of NMDA responses (0.96±0.23 fold; n=5; FIG. 5C; p<0.05; Mann-Whitney test for average potentiation 12-19 min post treatment). In a separate experiment, we premixed thrombin with an irreversible inhibitor of thrombin that covalently modifies the fibrinopeptide catalytic site for cleavage of substrate (500 nM PPACK; Tapparelli et al., 1993, supra) before application to the slice. The average peak potentiation of the NMDA response after thrombin-PPACK application was also significantly reduced (1.28±0.17; n=8) compared to thrombin treatment (2.07±0.27-fold; n=21; FIG. 5D; Kruskal-Wallis ANOVA, Dunn post hoc test; p<0.05). Non-parametric statistical tests were used because of the non-normal distribution of peak fold potentiation by thrombin (FIG. 5D). These experiments confirm that the potentiation of NMDA receptor currents we observe are due to the proteolytic actions of thrombin on either a protease receptor such as PAR1 or some other substrate.

Because PAR1 is expressed in the CA1 region and is known to couple to the G_{Q} family of G-alpha proteins, which can stimulate phosphoinositide hydrolysis, increase intracellular Ca²⁺, and activate intracellular protein kinases, we tested whether the serine/threonine protein kinase inhibitor bisindolylmaleimide (BIS) could occlude thrombin potentiation of NMDA receptor function. When BIS (10 microM) was included in ACSF, potentiation of NMDA receptor currents by application of 30 nM (3 U/ml) thrombin was reduced to 1.20±0.13 fold (n=10, p<0.05; ANOVA; FIG. 5D), indicating that the potentiating effect of thrombin requires at least one functional protein kinase. NMDA responses were unchanged during preapplication of BIS, suggesting that BIS does not induce an inhibition of the NMDA response that is additive to potentiation.

Thrombin does not alter extracellular volume fraction in hippocampal slices In vivo injection of thrombin into the brain can cause edema and tissue swelling, raising the possibility that the potentiation we observe of NMDA receptor responses to agonist applied from a point source might reflect a change in the spatial diffusion profile of NMDA and glycine secondary to thrombin-induced changes in extracellular volume fraction. For example, any changes in the extracellular volume fraction that amplified the concentration of pressure ejected NMDA in the narrow clefts or expanded the tissue volume reached by NMDA would potentiate the NMDA response. To ensure that the diffusional characteristics of pressure applied NMDA are not influenced by thrombin treatment, we measured the effects of thrombin application on transmitted light, which has previously been shown to be a sensitive measure of extracellular volume fraction (Andrew and MacVicar, 1994, supra). 300 micron hippocampal slices were prepared and incubated in 0.5 micromolar TTX to abolish synaptic activity. Perfusion of these slices with solutions made hyperosmotic by addition of 30 mM mannitol reduced the transmitted light by 10% in both CA1 stratum radiatum and stratum pyramidale, consistent with expansion of the extracellular space. Conversely, hypoosmotic solutions produced a 20% increase in transmitted light consistent with induction of cell swelling. Treatment of slices with 3-7 U/ml of thrombin for 10-20 minutes produced no significant change in the intensity of transmitted light (1) in either stratum pyramidale (I/Iₒ=1.00±0.01) or stratum radiatum (I/Iₒ=1.01±0.01) compared to untreated slices. These data suggest that neither thrombin activation of PAR1 nor cleavage of other substrates significantly alters extracellular volume fraction in acute slices. We interpret these data to suggest that the potentiation of NMDA receptor responses that we observe following activation of PAR1 does not reflect a thrombin-induced change in the temporal-spatial diffusion profile of NMDA/glycine released into or above the tissue from our pressurized pipette.

Thrombin proteolysis of the NR1 subunit Because one defining feature of thrombin is its proteolytic action, we examined whether NMDA receptor protein subunits were substrates for thrombin cleavage. Incubation of brain membranes with thrombin for 1 hour at 37° C. resulted in the appearance of a new band that was 12 kD (n=10) lower in molecular weight than the parent band in immunoblots probed with an antibody to NR1 that recognizes the M3-M4 loop (mAb54.1; epitope between residues 660-811; Siegel et al., Regional, cellular, and ultrastructural distribution of N-methyl-D-aspartate receptor subunit 1 in monkey hippocampus. Proc. Natl. Acad Sci USA 91: 564-568, 1994). However, similar treatment of membranes did not induce any observable molecular weight shift of either the NR2A (n=8) or NR2B subunits (n=10; 3-300 U/ml). NR1 subunits in all brain regions tested were cleaved by high concentrations of thrombin (1-3 micromolar; 100-300 U/ml; n=5-7 for each region). The degree of cleavage by both high and low concentrations of thrombin varied across regions. Whereas cerebellar (n=7), cortical (n=5), and brainstem (n=5) NR1 subunits were insensitive to cleavage by 300 nM thrombin (30 U/ml), 30 nM thrombin (3 U/ml) cleaved 20% of the NR1 subunit in hippocampal membranes (n=7) and 50% of NR1 in striatal membranes (n=10), suggesting that receptors in these membranes are more sensitive to the effects of thrombin. In addition, 1-3 micromolar thrombin cleaved 100% or subunits in all brain regions except striatum, where the maximal observed cleavage of 52% suggests a thrombin resistant population of NR1 subunits exists. Thrombin-induced NR1 cleavage does not involve PAR1 receptor-activated second messenger systems since 0.1-1 mM PAR1 peptide agonist SFLLRN did not stimulate cleavage (n=3). In addition 100 ATU hirudin, a thrombin inhibitor, blocked cleavage of NR1 in striatal membranes by 30 nM thrombin (n=3), suggesting that the shift in the molecular weight of the NR1 subunit does not reflect the actions of contaminant proteases.

Recombinant NR1 subunits transiently expressed in HEK 293 cells were also sensitive to thrombin cleavage. 10-1000 U/ml of thrombin cleaved both homomeric NR1-1b receptors (n=9/10 experiments) and heteromeric NR1-1b/NR2B receptors (n=6/6 experiments). The appearance of a new NR1 band of reduced molecular weight in thrombin-treated brain membranes was detected using mAb54.1, which recognizes an epitope located near the M3 transmembrane region (Siegel et al., 1994, supra). In order to evaluate the location of the thrombin cleavage site, we assessed the effects of thrombin on recombinant NR1-1a subunit that contained a myc epitope inserted into the N-terminal after Asn50. Thrombin induced a similar shift in molecular weight for N-terminal myc-tagged NR1 as observed for the recombinant and native receptors detected with mAb54.1, suggesting that cleavage likely occurs near the C-terminal of the receptor. Consistent with this idea, deletion of the C-terminal portion of NR1-1b by replacement of the coding sequence starting at amino acid position 785 with a cDNA encoding HLEGPIL-stop abolished any detectable cleavage of the NR1 subunit. These data suggest that thrombin cleaves NR1 subunit somewhere near the C-terminal and were consistent with immunoblots performed using a C-terminal antibody. However, use of a C-terminal antibody recognized a larger cleavage product than predicted from our data. The reasons for the discrepancy in the molecular weight of the C-terminal NR1 cleavage product from brain membranes as detected with different antibodies are under investigation.

To test whether the potentiation of NMDA responses that we observed in hippocampal neurons treated with low concentrations of thrombin for short periods of time (12-20 minutes at 23° C.) was related to proteolysis of the NR1 subunit, the CA1 region was microdissected after electrophysiological experiments and analyzed by SDS/PAGE followed by immunoblot. Despite robust potentiation of the NMDA receptor response during the 15 minutes of 3 U/ml thrombin application, no NR1 cleavage product was observed in immunoblots of membranes obtained from the CA1 region microdissected immediately following the experiment. Similar results were obtained from 6 slices, which are consistent with the data described above that show only modest (˜20%) cleavage of hippocampal NR1 subunit by four times longer thrombin treatments (1 hour incubation at 37° C. with 3 U/ml thrombin). If cleavage of NR1 by 3 U/ml thrombin is linearly related to time and temperature-sensitive with a Q₁₀ of 2.5, we predict from the 20% cleavage of hippocampal NR1 following 1 hour at 37° C. that thrombin should cleave 1% of the NR1 protein following 15 minute treatment of our slices at 23° C., which is likely below our limit of detection. In addition, there was no significant difference in the response amplitude of recombinant NR1/NR2A and NR1/NR2B NMDA receptors expressed in Xenopus oocytes, which lack thrombin receptors that were treated either with buffer or identical thrombin concentrations and incubation conditions used in slice experiments (3 U/ml; 15 minutes; 23° C.). Incubation of oocytes with higher concentrations of thrombin (300-1000 nM) for 60 minutes reduced current response amplitude to 42±9% (n=7), compared to 78±5% (n=6; p<0.05, t-test) for buffer-treated controls, suggesting that thrombin can inhibit NMDA receptor function, presumably through cleavage of NR1. These and other results (see below) suggest that the thrombin-mediated potentiation of the NMDA current responses in CA1 hippocampal pyramidal neurons does not reflect NR1 proteolysis or direct interaction of thrombin with NMDA receptors.

Thrombin potentiation of NMDA receptor function is PAR1-dependent To evaluate the working hypothesis that thrombin activation of the protease receptor PAR1 leads to potentiation of NMDA receptor secondary to activation of intracellular signaling pathways, we first verified that hippocampal neurons contain functional protease receptors, which are known to couple to GQ family of G-alpha-proteins. We measured increases in fluorescence of the calcium sensitive dye Fluo-3 in response to 3 nM thrombin or 10 micromolar PAR1 peptide agonist SFLLRN. This peptide matches the new N-terminal of PAR1 that is revealed by thrombin cleavage at Arg41, and is thought to act as a tethered activator of the receptor. Experiments were performed on cultured neurons in the presence of 0.5 micromolar TTX to reduce the synaptic activity and 50 micromolar APV to eliminate the possibility that thrombin potentiation of tonically active NMDA receptors might increase intracellular Ca²⁺. Images were acquired while the following sequence of solutions were perfused into the chamber: TTX/APV, TTX/APV/thrombin or SFLLRN, TTX/APV, TTX/NMDA/glycine. Both thrombin and the PAR1 peptide agonist SFLLRN elicited a robust increase in the Fluo-3 fluorescence in both the soma and dendrites of a subset of neurons, suggesting that these treatments increased intracellular Ca²⁺. The thrombin inhibitor PPACK (50 nM) reduced the percentage of neurons responding to thrombin and the response magnitude to control levels (1.1+0.04 fold; n=46; FIG. 5B). Because we could detect no proteolysis of NR1 in the slices we studied, the most likely explanation for the potentiation we observe following thrombin treatment was that thrombin activated PAR receptors on CA1 pyramidal cells. To directly evaluate this possibility we tested whether the specific PAR1 agonist peptide SFLLRN, which mimics the new N-terminal on PAR1 revealed following thrombin cleavage after Arg41, could also potentiate NMDA receptor responses in CA1 pyramidal cells in acute hippocampal slices. Application of 30 micromolar PAR agonist SFLLRN (10×EC₅₀) to the slice produced a 1.76±0.19-fold (n=9) potentiation of the response to pressure applied NMDA plus glycine, which was significantly different from that observed in buffer-treated slices (1.05+0.03-fold potentiation; n=8; p<0.01 Mann-Whitney test). These data are consistent with the idea that thrombin potentiates NMDA receptors through activation of a protease receptor, most likely PAR1.

To further evaluate the role of PAR1 activation in thrombin-induced potentiation of NMDA receptor responses, we studied the effects of thrombin application on hippocampal CA1 pyramidal cells from wild-type mice as well as mice engineered to lack the full length PAR1 gene (Connolly et al., 1996, supra). Application of 3 U/ml of thrombin caused a robust potentiation of responses to pressure-applied NMDA plus glycine in wild-type C57B1/6 mice that was significantly different at all times from that observed in control experiments in which ACSF was applied. However, application of thrombin to PAR1−/− C57B1/6 mice did not significantly increase NMDA receptor responses. Series and membrane resistance were monitored throughout the experiment, and showed only modest changes that could not account for the different effects of thrombin. As in rat hippocampal slices, NMDA receptor response in wild-type and PAR1−/− mice were abolished by 100 micromolar APV (n=9) following thrombin treatment, suggesting that any potentiation observed reflected NMDA receptor activation rather than sensitization of the cells to glycine or pressure. Whereas these experiments provide direct evidence for the involvement of PAR1 in thrombin-induced potentiation of NMDA receptor function, modest levels of thrombin potentiation of NMDA receptor responses still develop slowly in PAR1-/- mice, becoming significant only after thrombin application. This latent potentiation in PAR1−/− mice raises the possibility that thrombin cleavage of as yet uncharacterized PARs or other signaling substrates in hippocampal neurons might lead to modest levels of NMDA receptor potentiation observed in PAR1−/− mice.

Potentiation of recombinant NMDA receptors by activation of the thrombin receptor PAR1 To test whether PAR1 activation can directly alter NMDA receptor function in recombinant systems, we injected Xenopus oocytes with cRNA encoding NMDA receptor subunits alone or together with cRNA encoding the PAR1 receptor. The oocytes were placed under two-electrode voltage clamp to record agonist-evoked currents at a membrane holding potential of −30 mV in Mg²⁺-free solutions. Recordings were made in a paired fashion such that two oocytes, one of which was co-injected with PAR1, were recorded from simultaneously with the same solutions. The presence of functional PAR1 receptor was confirmed by observation of the Ca²⁺-activated Cl current during a 2-3 minute application of 0.03-3 U/ml (300 pM-30 nM) of thrombin. This inward current is observed when Ca²⁺ is released from intracellular stores following PAR1 activation of phosphoinositol-linked signaling. NMDA receptor responses in oocytes that were not coinjected with PAR1 cRNA (n=19) were not potentiated by thrombin application, whereas oocytes that were injected with PAR1 cRNA typically displayed thrombin-induced potentiation of the NMDA receptor-mediated current (n=23). Potentiation of NR1-1a/NR2A receptors coexpressed with PAR1 was observed after treatment with 300 pM (1.37±0.08 fold; n=7), 3 nM (1.68±0.11 fold; n=6), or 30 nM thrombin. Low concentrations of thrombin (0.3-3 nM) that were capable of evoking PAR1-mediated potentiation of NMDA receptor function did not produce any cleavage of NR1 subunit, further suggesting that the potentiation we observe is independent of thrombin proteolysis of NR1.

To confirm that activation of PAR1 leads to NMDA receptor current potentiation, we tested whether the PAR1 activating peptide (10-30 micromolar SFLLRN) could potentiate recombinant NMDA receptor responses in oocytes coinjected with PAR1 cRNA. Oocytes expressing NR1-1a/NR2B NMDA receptor subunits but not PAR1 had a ratio of the NMDA receptor-mediated current after thrombin application to the current response before thrombin application of 0.87±0.04 (n=9). By contrast, oocytes expressing the same NMDA receptor subunits and PAR1 had a significantly larger ratio after SFLLRN stimulation of PAR1 (1.41±0.11 fold potentiation; p<0.01; paired t-test; n=9). Similar results were found for SFLLRN potentiation of NR1-1a/NR2A receptors (1.85±0.1 fold potentiation; n=6). These data showing SFLLRN potentiation of recombinant NMDA receptor responses rule out the possibility that thrombin cleavage of substrates other than PAR1 might contribute to the observed NMDA receptor potentiation. To evaluate the subunit dependence of PAR1 potentiation of NMDA receptor responses, we co-injected oocytes with a variety of NR1 subunit combinations. Several NR1 splice variants co-expressed with NR2B that were tested for their sensitivity to PAR1 potentiation of receptor function. The NR1-4aΔstop mutation eliminates 22 carboxy terminal amino acids included in the NR1-4a subunit as a result of a frame shift associated with the use of an alternative splice site in exon22. Receptors containing all splice variants tested were significantly potentiated by PAR1 activation.

The NR2 subunit had a more marked effect on PAR1 potentiation of NMDA receptor function. Oocytes coinjected with NR1-1 a and either the NR2A or NR2B subunit were potentiated by thrombin activation of PAR1. Coexpression of NR3A with NR1-1a/NR2A or NR1-1a/NR2B subunits did not occlude PAR1-mediated potentiation. However the amplitude of NMDA receptor responses in oocytes coinjected with NR1-1a and either NR2C or NR2D was unaffected by thrombin activation of PAR1. These results suggest that only receptors that contain NR1/NR2A or NR1/NR2B subunits can be potentiated by PAR1 activation. In addition, these data are consistent with the idea that NR1 splicing alone does not dominate the molecular determinants of PAR1 potentiation in our Ba²⁺ containing recording solution.

The potentiation of NMDA receptor responses by thrombin activation of PAR1 was independent of voltage over the range −80 to +30 mV (n=6). The potentiation we observe occurred for responses to maximal concentrations of glutamate and glycine, eliminating the possibility that potentiation reflects an increase in the glutamate or glycine EC₅₀. PAR1 potentiation could also be observed for NR1-1 a(C798A)/NR2B receptors (1.35±0.07; n—7; p<0.05 by t-test) compared to control oocytes lacking PAR1 (0.98±0.05; n=5), which rules out any contribution to the potentiation we observe of changes in reduction/oxidation state of the disulfide linkage in NR1. In addition, PAR1 potentiation of recombinant NMDA receptor responses does not significantly alter the ratio of current responses at pH 7.6 and pH 6.8, suggesting that PAR1 activation does not relieve tonic proton inhibition. This result is consistent with the lack of effect of alternate splicing of NR1 exon5 on PAR1 potentiation. Potentiation of NR2A containing receptors was observed both in the absence and the presence of 10 micromolar EDTA (2.01+0.17 fold; n=12), eliminating any contribution of PAR1-mediated relief of tonic inhibition by contaminant Zn²⁺ in our recording solutions. PAR1-mediated potentiation did not reduce Mg²⁺-block of recombinant NMDA receptors. The modest enhancement of Mg²⁺ blockade following thrombin activation of PAR1 for NR1-1a/NR2B/NR3A receptors was not significantly different from a modest enhancement of Mg⁺ potentiation observed in thrombin treated control oocytes that did not express PAR1 (n=7), suggesting it was not linked to PAR1 activation.

Thrombin potentiation of neuronal NMDA receptor function is voltage-dependent Hippocampal NMDA receptors are under strong voltage dependent block by extracellular Mg²⁺, as determined by the current-voltage curve from hippocampal CA1 pyramidal cells. To investigate whether the thrombin-induced potentiation of neuronal NMDA receptors is independent of voltage-dependent Mg²⁺ blockade, we compared the ratio of NMDA-evoked whole cell currents recorded at −70 and −40 mV in control cells. We found that the ratio of current recorded at −70 mV to −40 mV was significantly larger after thrombin treatment when compared to pretreatment control (n=22; Wilcoxon rank sum test; p<0.01). Moreover, in 16/21 CA1 pyramidal cells examined, the potentiation of NMDA receptor responses by thrombin was larger at −70 mV. Average peak potentiation was 2.10±0.29-fold at −70 mV and 1.48±0.11-fold at 40 mV in 1.4 mM Mg²⁺ (n=19; Wilcoxon rank sum test; p<0.01). Interestingly, the magnitude of potentiation observed at −40 mV was virtually identical to that observed in Xenopus oocytes in the absence of Mg²⁺. We interpret these data to suggest that there may be an additional voltage-dependent component to the potentiation of neuronal NMDA receptors at hyperpolarized potentials following thrombin-activation of PAR1 that is not present in Xenopus oocytes. One potential explanation of this result is that PAR1 activation modestly reduces the voltage-dependent Mg²⁺ block of neuronal NMDA receptors in addition to potentiating receptor function in a voltage-independent fashion. An alternative explanation for the voltage-dependence of PAR1 potentiation of neuronal receptors is that PAR1-linked modification or thrombin proteolysis of other channels or membrane proteins reduces our ability to keep the dendrites under voltage control. The resulting reduction in holding potential of distal dendrites during NMDA receptor activation might modestly reduce NMDA receptor block by extracellular Mg²⁺, causing an apparent supplemental potentiation of the NMDA receptor response. Although we can detect no thrombin-induced changes in membrane resistance and have blocked some K⁺, Na⁺, and Ca²⁺ channels with Cs⁺, QX-314, and nifedipine, the complex electrotonic structure of CA1 hippocampal pyramidal cells makes it difficult to dismiss this alternative possibility.

Incorporation by Reference

Throughout this application, various publications, patents, and/or patent applications are referenced in order to more fully describe the state of the art to which this invention pertains. The disclosures of these publications, patents, and/or patent applications are herein incorporated by reference in their entireties, and for the subject matter for which they are specifically referenced in the same or a prior sentence, to the same extent as if each independent publication, patent, and/or patent application was specifically and individually indicated to be incorporated by reference.

Other Embodiments

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of treating a patient with an injury of the central nervous system caused by a trauma to the head or spinal cord, comprising administering to the patient a therapeutically effective amount of a PAR1/thrombin receptor antagonist, thereby treating the injury to the central nervous system caused by the trauma to the head or spinal cord.

2. The method of claim 1, wherein the injury is a penetrating injury.

3. The method of claim 1, wherein the injury is a crush injury, a compression injury, or a stretch injury.

4. The method of claim 1, wherein the injury is a blunt injury.

5. A method of treating a patient with a subarachnoid hemorrhage, comprising administering to the patient a therapeutically effective amount of a PAR1/thrombin receptor antagonist, thereby treating the subarachnoid hemorrhage.

6. A method of inhibiting reactive gliosis and/or gliotic scar formation in a patient in need thereof, comprising:
a) identifying the patient as being in need of administration of a therapeutically effective amount of a PAR1/thrombin receptor antagonist for the purpose of inhibiting gliosis and/or gliotic scar formation, and
b) administering to the patient a therapeutically effective amount of the PAR1/thrombin receptor antagonist, thereby inhibiting the gliosis and/or gliotic scar formation.

7. A method of reducing damage to the brain as a result of intracerebral bleeding, comprising:
a) identifying a patient as being in need of administration of a therapeutically effective amount of a PAR1/thrombin receptor antagonist for the purpose of reducing damage to the brain as a result of intracerebral bleeding, and
b) administering to the patient a therapeutically effective amount of the PAR1/thrombin receptor antagonist, thereby reducing damage to the brain as a result of intracerebral bleeding.

8. The method of claim 7, wherein the intracerebral bleeding is the result of a brain aneurysm, ruptured arteriovenous malformation, or traumatic brain injury.

9. A method of reducing brain damage or neuron death that results from a seizure, comprising administering, to a patient who is having a seizure, who has recently had a seizure, or who is at risk for having a seizure, a therapeutically effective amount of a PAR1/thrombin receptor antagonist, thereby reducing brain damage that results from a seizure.

10. The method of claim 9, wherein the seizure is associated with chronic epilepsy, idiopathic epilepsy, post-traumatic epilepsy, or status epilepticus.

11. A method of reducing neuron death that is caused by NMDA receptor overactivation in a patient, comprising:
a) identifying a patient as being in need of administration of a therapeutically effective amount of a PAR1/thrombin receptor antagonist for the purpose of reducing neuron death that is caused by NMDA receptor overactivation, and
b) administering to the patient a therapeutically effective amount of the PAR1/thrombin receptor antagonist, thereby reducing neuron death that is caused by NMDA receptor overactivation in the patient.

12. A method of treating a neuropathological disease, condition, or injury, comprising:
a) identifying a subject as being in need of treatment to reduce neuron death by inhibition of PAR1 receptor activity on neurons, or as being in need of treatment to reduce reactive gliosis and/or glial scar formation by inhibition of PAR1 receptor activity on glia, and
b) administering to the patient a therapeutically effective amount of a PAR1/thrombin receptor antagonist, thereby treating the neurological disease, condition, or injury.

13. The method of claim 12, wherein the neuropathological disease, condition, or injury is head trauma, spinal cord trauma, brain aneurysm, intracerebral bleeding, subarachnoid hemorrhage, seizure, idiopathic epilepsy, chronic epilepsy, post-traumatic epilepsy, status epilepticus, Alzheimer's disease, Parkinson's disease, lower spinal motor neuron disease, upper motor neuron disease, amyotrophic lateral sclerosis, multiple sclerosis, or demyelinating disease.

14. The method of claim 12, wherein the neuropathological disease, condition, or injury is occlusive stroke, hemorrhagic stroke, thrombosis, stenosis, or transient ischemic attack.

15. A method of treating a patient during or following global ischemia as a result of cardiac surgery or pulmonary surgery, comprising administering to the patient a therapeutically effective amount of a PAR1/thrombin receptor antagonist for the purpose of:
a) reducing brain damage, gliosis, gliotic scar formation, and/or neuronal death resulting from global ischemia resulting from cardiac surgery or pulmonary surgery, and/or
b) facilitating recovery from neurological deficit resulting from cardiac surgery or pulmonary surgery,
thereby treating a patient following or during global ischemia as a result of cardiac surgery or pulmonary surgery.

16. A method of treating a patient suffering from a hypoxic condition, comprising administering to the patient a therapeutically effective amount of a PAR1/thrombin receptor antagonist for the purpose of:
a) reducing the associated brain damage, gliosis, gliotic scar formation, and/or neuronal death resulting from the hypoxic condition, and/or
b) facilitating recovery from neurological deficit resulting from the hypoxic condition,
thereby treating the patient suffering from a hypoxic condition.

17. The method of claim 16, wherein the patient suffers from anemic hypoxia, hypoxic hypoxia, anoxic hypoxia, histopathic hypoxia, or hypoglycemic hypoxia.

18. A method of treating a patient with hypoglycemia, comprising administering to the patient a therapeutically effective amount of a PAR1/thrombin receptor antagonist for the purpose of:
a) reducing brain damage, gliosis, gliotic scar formation, and/or neuronal death resulting from hypoglycemia, and/or
b) facilitating recovery from neurological deficit resulting from hypoglycemia, thereby treating the patient with hypoglycemia.

19. A method of treating a patient with a bacterial, viral, fungal, or parasite infection of the central nervous system, comprising administering to the patient a therapeutically effective amount of a PAR1/thrombin receptor antagonist as an adjunct treatment for the purpose of:
a) reducing brain damage, gliosis, gliotic scar formation, and/or neuronal death resulting from the infection, and/or
b) facilitating recovery from neurological deficit resulting from the infection, thereby treating the patient with a bacterial, viral, fungal, or parasite infection of the central nervous system.

20. A method of treating a patient with a prion infection, comprising administering to the patient a therapeutically effective amount of a PAR 1/thrombin receptor antagonist as an adjunct treatment for the purpose of:
a) reducing brain damage, gliosis, gliotic scar formation, and/or neuronal death resulting from the prion infection, and/or b) facilitating recovery from neurological deficit resulting from the prion infection,
thereby treating the patient with a prion infection.

20. The method of claim 20, wherein the patient suffers from Creutzfeldt-Jakob disease.

21. A method of treating a patient with increased intracranial pressure, comprising administering to the patient a therapeutically effective amount of a PAR1/thrombin receptor antagonist for the purpose of:
a) reducing brain damage, gliosis, gliotic scar formation, and/or neuronal death resulting from the increased intracranial pressure, and/or
b) facilitating recovery from neurological deficit resulting from the increased intracranial pressure, thereby treating the patient with increased intracranial pressure.
